(19) United States
(12) Patent Application Publication
(10) Pub. No.: US 2023/0033299 A1
(43) Pub. Date: Feb. 2, 2023

(54) Methods of Treating Pain Conditions and Compositions Related Thereto

(71) Applicant: NxGen Medicine Inc., Belmont, CA (US)

(72) Inventors: Stephan GEHRKE, Belmont, CA (US); Hui-Lin PAN, Pearland, TX (US)

(21) Appl. No.: 17/621,435

(22) PCT Filed: Jun. 25, 2020
(86) PCT No.: PCT/US2020/039676

§ 371 Date: Dec. 21, 2021

Related U.S. Application Data
(60) Provisional application No. 62/880,382, filed on Jul. 30, 2019.
(60) Provisional application No. 62/949,626, filed on Dec. 18, 2019.

(51) Int. Cl. A61K 48/00; A61P 25/02

(52) U.S. Cl. CPC A61K 48/005; A61P 25/02

(57) ABSTRACT

Methods are provided for treating a subject with a pain condition. Aspects of the methods include administering a gene therapy to the subject and/or a therapeutically effective amount of a composition that includes a gene therapy vector. Aspects of the vectors may include a nucleic acid sequence encoding a K-Cl cotransporter 2 (KCC2) polypeptide, including e.g., full-length and modified versions thereof. Methods are also provided for treating a subject by editing an endogenous KCC2 locus of the subject to encode a modified KCC2 polypeptide. Methods of detecting the presence of a pain condition are also provided, including where a pain condition detected in such methods is treated according to the methods described herein. Also provided are compositions, such as compositions including a gene therapy vector, such as a lentiviral vector, that includes a viral backbone nucleic acid comprising a sequence encoding a full-length human KCC2 polypeptide or a nucleic acid sequence encoding a modified KCC2 polypeptide.

### CROSS-REFERENCE TO RELATED APPLICATIONS

Pursuant to 35 U.S.C. § 119(e), this application claims priority to the filing dates of U.S. Provisional Patent Application Ser. No. 62/880,382 filed Jul. 30, 2019 and 62/949,626 filed Dec. 18, 2019; the disclosures of which applications are incorporated herein by reference.

### INTRODUCTION

When persistent and left untreated, pain conditions can generate numerous pathologies that often approximate, or even surpass, the direct detrimental effects of the underlying etiology of the pain condition or the original injury from which the pain condition arose. Pathophysiological changes as a result of a pain condition can be long lasting and can involve structural and functional alterations such that pain ceases to be symptomatic of the initial cause and becomes an entirely separate condition. This can have both physical and psychological consequences for patients, as well as a substantial economic impact due to increased costs of health care and lost productivity (Fine P G. Pain Medicine (2011) 12: 996-1004).

Pain conditions, both chronic and non-chronic, are highly prevalent, with chronic conditions, by some estimates, affecting approximately one third of the adult population in the United States. Neuropathic pain in particular is estimated to afflict some 7-8% of the worldwide population. Peripheral nerve disorders are also common conditions, with the most common being diabetic neuropathies that affect both type 1 and 2 diabetics. As a whole, peripheral neuropathies are estimated to affect 2.4% of the population. Pain from peripheral neuropathy is a serious symptom for many patients, described as presenting in various different ways, including e.g., a dull aching sensation, an intense burning sensation or, occasionally, as intermittent lancinating pulses of pain. The skin of patients may, in some instances, be hypersensitive to tactile stimulation, including touch from generally non-painful objects, such as soft fabrics, and pain while performing usually pain-free activities, such as standing or walking. Some patients note a condition termed allodynia, which is an exaggerated painful sensation resulting from any stimulus to the affected area.

Despite the high prevalence of pain conditions in the U.S. and populations worldwide, avenues of treatment are often problematic due, at least in part, to the habit-forming nature of many pain medications and various ways in which pain conditions present. In fact, in the U.S., at least in part due to an ongoing epidemic of opioid abuse, pain treatments are rapidly becoming less, rather than more, available.

### SUMMARY

Methods are provided for treating a subject with a pain condition. Aspects of the methods include administering a gene therapy to the subject and/or a therapeutically effective amount of a composition that includes a gene therapy vector. Aspects of the vectors may include a nucleic acid sequence encoding a K-Cl cotransporter 2 (KCC2) polypeptide, including e.g., full-length and modified versions thereof. Methods are also provided for treating a subject by editing an endogenous KCC2 locus of the subject to encode a modified KCC2 polypeptide. Methods of detecting the presence of a pain condition are also provided, including where a pain condition detected in such methods is treated according to the methods described herein. Also provided are compositions, such as compositions including a gene therapy vector, such as a lentiviral vector, that includes a viral backbone nucleic acid comprising a sequence encoding a full-length human KCC2 polypeptide or a nucleic acid sequence encoding a modified KCC2 polypeptide.

### BRIEF DESCRIPTION OF THE FIGURES

FIG. 1 provides a schematic depiction of a cross-section of a mammalian spinal cord, showing dorsal horn target areas and an infusion catheter in the subarachnoid space.

FIG. 2 provides a schematic depiction of a catheter inserted such that the caudal tip is present at a target location.

FIG. 3 provides a schematic depiction of a catheter inserted and withdrawn such that the caudal tip is positioned to deliver agent across two target locations.

FIG. 4 provides exemplary experimental timelines employed in the rat Spinal Nerve Ligation (SNL) model study described.

FIG. 5 demonstrates pain reduction in SNL rats administered cytomegalovirus (CMV)- or human synapsin 1 (hSyn1)-driven full-length human KCC2 vectors as assessed using a behavior test as described.

FIG. 6 shows differences in pain suppression observed for each of the full-length, truncated and mutant KCC2 viral vector forms administered in the SNL rat model.

FIG. 7 provides an exemplary experimental timeline employed in the rat Streptozotocin (STZ)-induced neuropathic pain model study described.

FIG. 8 demonstrates significantly increased blood glucose levels in streptozotocin treated rats, confirming STZ-mediated toxicity of insulin producing beta cells in the model.

FIG. 9 demonstrates the effectiveness of CMV-driven full-length hKCC2 viral vector in suppressing diabetic neuropathy in the rat STZ model.

FIG. 10 shows significant suppression of pain in left and right limbs of STZ diabetic rats administered CMV-driven full-length hKCC2 viral vector.

FIG. 11 shows significant suppression of free hKCC2 protein levels in lumbar CSF samples taken from pain patients, as compared to healthy controls.

FIG. 12 provides individual results showing decreased levels of free hKCC2 in CSF samples taken from pain patients, as compared to healthy controls, and quantitation related thereto.

### DEFINITIONS

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “gene” refers to a particular unit of heredity present at a particular locus within the genetic component of an organism. A gene may be a nucleic acid sequence, e.g., a DNA or RNA sequence, present in a nucleic acid genome, a DNA or RNA genome, of an organism and, in some instances, may be present on a chromosome. Typically, a gene will be a DNA sequence that encodes for an mRNA that encodes a protein. A gene may be comprised of a single exon and no introns or multiple exons and one or more introns. One of two or more identical or alternative forms of a gene present at a particular locus is referred to as an “allele” and, for example, a diploid organism will typically have two alleles of a particular gene. New alleles of a particular gene may be generated either naturally or artificially through natural or induced mutation and propagated through breeding or cloning.

“Operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

“Heterologous,” as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and endogenous sequences, with or without N-terminal methionine residues; tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or non-proteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells and/or introduced into a heterologous organism, e.g., using a vector, since at least one component of the polypeptide's natural environment will not be present. In some instances, isolated polypeptide will be prepared by at least one purification step.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluids, such as cerebrospinal fluid, and tissue samples.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Life Technologies, Inc., Sigma-Aldrich, and Takara Bio USA, Inc.

### DETAILED DESCRIPTION

Methods are provided for treating a subject with a pain condition. Aspects of the methods include administering a gene therapy to the subject and/or a therapeutically effective amount of a composition that includes a gene therapy vector. Aspects of the vectors may include a nucleic acid sequence encoding a K-Cl cotransporter 2 (KCC2) polypeptide, including e.g., full-length and modified versions thereof. Methods are also provided for treating a subject by editing an endogenous KCC2 locus of the subject to encode a modified KCC2 polypeptide. Methods of detecting the presence of a pain condition are also provided, including where a pain condition detected in such methods is treated according to the methods described herein. Also provided are compositions, such as compositions including a gene therapy vector, such as a lentiviral vector, that includes a viral backbone nucleic acid comprising a sequence encoding a full-length human KCC2 polypeptide or a nucleic acid sequence encoding a modified KCC2 polypeptide.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods are provided for treating a subject for a pain condition, including e.g., where such a subject may be a mammalian subject, such as a rodent (e.g., a mouse, a rat, a guinea pig, a hamster, a gerbil, etc.), a swine, a canine, a feline, a lagomorph, a non-human primate, or a human. Subjects treated using the herein described methods may have a pain condition or have an elevated risk of developing a pain condition. In some instances, the pain condition may be identified or detected in the subject, including but not limited to e.g., where such identification or detection includes employing the detection methods described in more detail below. Various pain conditions are treatable through use of the herein described methods, including but not limited to e.g., those exemplary pain conditions described below.

Methods of the present disclosure may include administering a therapeutically effective amount of a composition to e.g., a central nervous system location, e.g., the spinal cord, of the subject, thereby treating the subject for the pain condition, where such compositions may vary. Compositions useful in the herein described methods will generally include one or more gene therapy vectors that include one or more nucleic acids encoding one or more K-Cl cotransporter 2 (KCC2) polypeptides, where gene therapy may be viral or non-viral gene therapy. Useful KCC2 polypeptides, described in more detail below, may include full-length KCC2 polypeptides and modified KCC2 polypeptides, including but not limited to, e.g., human full-length KCC2 polypeptides and modified human KCC2 polypeptides. Useful modified KCC2 polypeptides, include KCC2 polypeptides that have been truncated, modified to include one or more amino acid substitutions, or both.

Administration of a gene therapy vector composition of the present disclosure will generally cause the subject to which the gene therapy vector is administered to express a KCC2 polypeptide, including full-length and/or modified KCC2 polypeptides, encoded by a nucleic acid of the vector. In some instances, where a modified KCC2 polypeptide is employed, administering a gene therapy agent, e.g., using a viral or non-viral gene therapy approach, as desired, to the subject may be effective to cause the subject to express an effective amount of a modified KCC2 polypeptide having enhanced activity relative to wild-type KCC2. In some instances, where a full-length KCC2 polypeptide is employed, administering a gene therapy agent to the subject may be effective to cause the subject to express an effective amount of the full-length KCC2 polypeptide. A “therapeutically effective amount” or “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of vector (e.g., KCC2 encoding viral vector, and the like) and/or compositions (e.g., KCC2 encoding vector compositions (e.g., full-length and/or modified KCC2 vector compositions)) is an amount that is sufficient, when administered to (e.g., infused into, delivered intrathecally or intraspinally, subpial, intra cisterna magna, etc.) the individual, to palliate, ameliorate, stabilize, reverse, prevent, slow or delay the progression of a pain condition of the subject. Administration of an effective amount of a vector may, for example, result in reducing the subject's pain level, reducing the subject's pain frequency, reducing the subject's pain duration, or some combination thereof. In some instances, an effective amount reduces one or more symptoms of a pain condition (including but not limited to, e.g., numbness, prickling or tingling, sensitivity to touch, lack of coordination and falling, muscle weakness, paralysis, etc.).

In some instances, a therapeutically effective dose, whether delivered in a single administration or multiple administrations, of a vector may remain effective for an extended period of time. The extended time period during which an administered therapeutically effective dose of a vector may remain effective will vary and may range from days to weeks to months including but not limited to, e.g., 1 week to 2 weeks, 2 weeks to 3 weeks, 3 weeks to 1 month, 1 week to 3 weeks, 2 weeks to 1 month, 1 week to 1 month, 1 month to 2 months, more than 2 months, etc.

The terms “treatment”, “treating”, “treat” and the like are used herein to generally refer to obtaining a desired pharmacologic and/or physiologic effect. The effect can be prophylactic in terms of completely or partially preventing a disease (wherein the term “disease” may encompass a pain condition) or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with already having a pain condition) as well as those in which prevention is desired (e.g., those at elevated risk of developing a pain condition, those having one or more risk factors for developing a pain condition, etc.).

A therapeutic treatment is one in which the subject is inflicted prior to administration and a prophylactic treatment is one in which the subject is not inflicted prior to administration. With respect to intermittent conditions (such as a relapsing-remitting pain condition), a prophylactic treatment may include a treatment administered to a subject with a diagnosed condition in a remitting state, e.g., to prevent a relapse of the condition or to prevent the reoccurrence of one or more symptoms of the condition. In some embodiments, the subject has an increased likelihood of becoming inflicted or is suspected of having an increased likelihood of becoming inflicted (e.g., relative to a standard, e.g., relative to the average individual), in which case the treatment can be a prophylactic treatment.

Aspects of the invention include administering a gene therapy agent, such as described below, to a subject. The gene therapy agent may be administered using any convenient approach, where such approaches include but not are not limited to: viral and non-viral gene therapy, gene editing (insertional mutagenesis of the host genome (e.g., as described above) and/or insertion of expression cassette into the host genome), coding DNA and RNA transduction, etc. Where desired vectors may be employed to deliver the gene therapy agent to target cells. Vectors that find use in such applications include, for example, DNA (non-viral) vectors and viral vectors. Circular plasmid DNA can enter cells in its naked form, or being covered with chemicals to enhance stability and delivery efficiency. Viral vectors take advantage of the infectious nature and gene-shuttling capability of certain viruses Both types of vectors can directly deliver genes into human body. DNA (non-viral) vectors. A therapeutic gene expression cassette is typically composed of a promoter that drives gene transcription, the transgene of interest, and a termination signal to end gene transcription. Such an expression cassette can be embedded in a plasmid (circularized, double-stranded DNA molecule) as delivery vehicle. Plasmid DNA (pDNA) can be directly injected by a variety of injection techniques, including hydrodynamic injection. To help negatively charged pDNA molecules penetrate the hydrophobic cell membranes, chemicals including cationic lipids and cationic polymers have been used to condense pDNA into lipoplexes and polyplexes, respectively. These nanoparticles shield pDNA from nuclease degradation in extracellular space and facilitate entry into target cells. Viral vectors. Many mammalian viruses have been explored as gene delivery vectors. Replacing most of the viral genes with a therapeutic gene cassette, and meanwhile retaining signal sequences that are essential for in vitro replication and packaging in producer cell lines formulate the common theme of viral vector genome engineering. Viral vector production commonly employs a trans-packaging system in cell culture, requiring the co-existence of one to four components. Vectors based on gammaretrovirus, lentivirus, adenovirus (AdV), adeno-associated virus (AAV) and herpes simplex virus (HSV) are among the most widely used viral vectors. Gammaretrovirus and lentivirus are both retrovirus, which is characterized by an RNA genome, and utilizing virus-derived reverse transcriptase and integrase to insert their proviral complementary DNA into the host genome. Gammaretrovirus can only transduce replicating cells, whereas lentivirus can also transduce non-replicating cells, which makes lentiviral vector more favorable in many gene therapy settings. Vector development based on these two viruses has greatly benefited from engineering their envelope glycoproteins that are amenable to modification for specific tissue and/or cell tropisms. For targeted gene delivery to a specific cell type, retroviral vectors can be pseudotyped with a viral glycoprotein that binds to a specific membrane receptor of that cell type. Furthermore, a viral glycoprotein can be fused with a ligand protein or antibody that recognizes cell type-specific surface molecules, providing a versatile way of cell type-specific gene delivery. Integration into host genome, the distinctive feature of retroviral vectors, ensures the stability of transgene and persistent transgene expression in daughter cells following genome replication and cell division. Most retroviral vectors are based on a self-inactivating (SIN) vector design. In SIN vector design, the enhancer/promoter sequences in LTR are removed, thus greatly reducing the likelihood of activating oncogenes. Other approaches aiming at reducing the risk of insertional mutagenesis include the development of integration-deficient lentiviral vectors by mutating the integrase, and site-directed integration using zinc finger nuclease. In contrast to retrovirus, AdV contains a DNA genome that episomally resides in host nucleus, which prevents insertional mutagenesis. AdV is able to transduce a broad range of quiescent and proliferating human cells. Currently, the most commonly used AdV vectors are derived from AdV serotype 5 (AdV5). AAV is a group of small, simple, helper-dependent, nonpathogenic, and single-stranded DNA viruses. Recombinant AAV (rAAV) vector carrying inverted terminal repeats as the only viral component entered the gene therapy arena much later than retroviral and AdV vectors, but have quickly gained popularity. For rAAV vectors, it is largely the capsid that determines the tropism and transduction profile in different cell types. Tropism of several natural AAV capsids has been well characterized in mouse and larger animal models. In addition to relying on natural diversity, AAV capsids can be decorated by peptides or “shuffled” to generate novel capsids that suit specific needs. Similar to AdV vector, rAAV vector can transduce both dividing and non-dividing cells, and its recombinant viral genome stays in host nucleus predominantly as episome. Expression of transposase mediates a cut-and-paste mechanism that efficiently inserts a designer transposon harboring a transgene cassette into host genome. The DNA transposon/transposase system can be delivered in vivo or ex vivo in the simple form of plasmid DNA. Instead of inserting extra DNA material into host genome, another approach to permanently correcting a diseased genome is through targeted genomic editing. Designer DNA endonucleases such as the CRISPR/Cas system can be engineered to cut genomic DNA in a sequence-specific manner, allowing for disruption or repair of that region. The genes orchestrating this process need only to be transiently expressed in cultured cells, whereas the mark in the genome is left permanent. Additional nucleic acid delivery protocols of interest include, but are not limited to: those described in U.S. patents of interest include U.S. Pat. Nos. 5,985,847 and 5,922,687 (the disclosures of which are herein incorporated by reference); WO/11092; Acsadi et al., New Biol. (1991) 3:71-81; Hickman et al., Hum. Gen. Ther. (1994) 5:1477-1483; and Wolff et al., Science (1990) 247: 1465-1468; etc.

In some instances, methods of treating a mammalian subject for a pain condition of the present disclosure may include administering to the subject a gene therapy agent effective to cause the subject to express an effective amount of a full-length KCC2 polypeptide. The gene therapy agent may include a sequence encoding the full-length KCC2 polypeptide. In some instances, a sequence encoding the full-length KCC2 polypeptide may be operably linked to a heterologous promoter, i.e., a promoter not naturally present in the host into which the gene therapy agent is introduced. In some instances, a sequence encoding the full-length KCC2 polypeptide may be operably linked to an additional copy of a promoter naturally present in the host into which the gene therapy agent is introduced. Accordingly, such additional copy may be not be naturally occurring and/or may not naturally be operably linked to a sequence encoding the full-length KCC2 polypeptide.

In some instances, methods of treating a mammalian subject for a pain condition of the present disclosure may include administering to the subject a gene therapy agent effective to cause the subject to express an effective amount of a modified KCC2 polypeptide having enhanced activity relative to wild-type KCC2. The gene therapy agent may include a sequence encoding the modified KCC2 polypeptide. In some instances, a sequence encoding the modified KCC2 polypeptide may be operably linked to a heterologous promoter, i.e., a promoter not naturally present in the host into which the gene therapy agent is introduced. In some instances, a sequence encoding the modified KCC2 polypeptide may be operably linked to an additional copy of a promoter naturally present in the host into which the gene therapy agent is introduced. Accordingly, such additional copy may be not be naturally occurring and/or may not naturally be operably linked to a sequence encoding the modified KCC2 polypeptide. In some instances, the modified KCC2 polypeptide may be operably linked to an endogenous promoter, including the endogenous KCC2 promoter.

Causing the subject to express the modified KCC2 polypeptide may be achieved in a variety of ways. For example, in some instances, expressing the modified KCC2 polypeptide may include expressing a heterologous KCC2 polypeptide encoded by the gene therapy agent. Put another way, in some instances, the modified KCC2 polypeptide may be introduced through the introduction of an expression cassette from which the modified KCC2 polypeptide is expressed.

In some instances, useful modified KCC2 polypeptides that may be expressed will vary and may include truncated and/or mutated KCC2 polypeptides, including e.g., a truncated KCC2 polypeptide, a KCC2 polypeptide having a 1906A substitution, a KCC2 polypeptide having a T934D substitution, a KCC2 polypeptide having a S937D substitution, a KCC2 polypeptide having a T1007A substitution, a truncated KCC2 polypeptide having a 1906A substitution, a truncated KCC2 polypeptide having a T934D substitution, a truncated KCC2 polypeptide having a S937D substitution, a truncated KCC2 polypeptide having a T1007A substitution, a KCC2 polypeptide having some combination of substitutions selected from 1906A, T934D, S937D and T1007A, a truncated KCC2 polypeptide having some combination of substitutions selected from 1906A, T934D, S937D and T1007A, and the like.

In some instances, expressing the modified KCC2 polypeptide may include editing an endogenous KCC2 locus of the subject to encode the modified KCC2 polypeptide. By “editing the endogenous locus” is generally meant, modifying the native locus of the gene present in the individual's genome such that the modified endogenous locus expresses a modified polypeptide as desired.

For example, in some instances, the endogenous KCC2 locus is edited to encode a modified KCC2 polypeptide comprising a desired modification, where such modifications may vary. Useful KCC2 locus modifications may include an N-terminal truncation (e.g., relative to a wild-type KCC2 polypeptide or the endogenous locus that is modified), one or more substitution mutations (e.g., relative to a wild-type KCC2 polypeptide or the endogenous locus that is modified). In some instances, only a single edit may be introduced, including e.g., a truncation or a substitution. In some instances, multiple edits (e.g., relative to a wild-type KCC2 polypeptide or the endogenous locus that is modified) may be introduced, including e.g., where a truncation and a substitution are both introduced, where multiple substitutions are introduced, or where a truncation and multiple substitutions are introduced. Where multiple substitutions are introduced the number of substitutions may vary, including but not limited to e.g., 2 or more, 3 or more, 4 or more, 2, 3, 4, etc.

In some instances, a locus may be edited to include a modified KCC2 polypeptide as described herein, including e.g., a truncated KCC2 polypeptide, a KCC2 polypeptide having a 1906A substitution, a KCC2 polypeptide having a T934D substitution, a KCC2 polypeptide having a S937D substitution, a KCC2 polypeptide having a T1007A substitution, a truncated KCC2 polypeptide having a 1906A substitution, a truncated KCC2 polypeptide having a T934D substitution, a truncated KCC2 polypeptide having a S937D substitution, a truncated KCC2 polypeptide having a T1007A substitution, a KCC2 polypeptide having some combination of substitutions selected from 1906A, T934D, S937D and T1007A, a truncated KCC2 polypeptide having some combination of substitutions selected from 1906A, T934D, S937D and T1007A, and the like.

Any convenient method of editing of the endogenous locus to achieve a locus encoding a desired modified KCC2 polypeptide may be employed. For example, in some instances, all or a portion of the coding sequence at the endogenous locus may be replaced with sequence encoding the modified KCC2 polypeptide or corresponding portion thereof. Methods of gene editing and/or replacement include but are not limited to e.g., those employing homology directed repair. In such methods, e.g., a nuclease may be employed to cleave the target locus and the modified copy may be introduced through a homologous recombination event. Accordingly, in such methods, a gene therapy vector may be provided that includes sequencing encoding the modified polypeptide, or the modified portion thereof, along with components sufficient to facilitate cleavage of the target locus and, optionally, one or more agents to enhance the processes of homologous recombination and/or one or reagents to assist in targeting the nuclease (such as e.g., a guide RNA).

Methods of modifying a target locus by homology directed repair will generally, but not necessarily, include the use of a nuclease for use in cleaving the target locus in order to facilitate or expedite the homology directed repair. In some embodiments, the target locus is contacted with a nucleic acid encoding the modified polypeptide, having homology arms that are homologous to targeted regions of the locus, and one or more nucleases.

Useful nucleases will vary and the selection of which may depend on e.g., the locus to be modified, the type of genome to be modified, the modified polypeptide-encoding nucleic acid to be introduced, and the like. Any convenient targeting nuclease may find use in the methods as described herein. In some instances, the subject nuclease may be administered as a nucleic acid encoding the nuclease, including e.g., where such nucleic acid sequence may be operably linked to a promoter driving expression of the nuclease.

In some instances, a methods of locus targeting may include the use of a Cas9 nuclease, including natural and engineered Cas9 nucleases. Useful Cas9 nucleases include but are not limited to e.g., Streptococcus pyogenes Cas9 and variants thereof, Staphylococcus aureus Cas9 and variants thereof, Actinomyces naeslundii Cas9 and variants thereof, Cas9 nucleases also include those discussed in PCT Publications Nos. WO 2013/176772 and WO2015/103153 and those reviewed in e.g., Makarova et al. (2011) Nature Reviews Microbiology 9:467-477, Makarova et al. (2011) Biology Direct 6:38, Haft et al. (2005) PLOS Computational Biology 1:e60 and Chylinski et al. (2013) RNA Biology 10:726-737, the disclosures of which are incorporated herein by reference in their entirety. In some instances, a non-Cas9 CRISPR nuclease may be employed, including but not limited to e.g., Cpf 1.

Cas9 nucleases are used in the CRISPR/Cas9 system of genomic DNA modification. In the CRISPR/Cas9 system a chimeric RNA containing the target sequence (i.e., the “guide RNA” or “small guide RNA (sgRNA)”, which collectively contains a crRNA and a tracrRNA) guides the Cas9 nuclease to cleave the DNA at a specific target sequence defined by the sgRNA. The synthesis of only a 20 bp guide RNA is required to program the nuclease. The specificity, efficiency and versatility of targeting and replacement of homologous recombination is greatly improved through the combined use of various homology-directed repair strategies and CRISPR nucleases (see e.g., Gratz et al. (2014) Genetics. 196(4)961-971; Chu et al. (2015) Nature. 33:543-548; Hisano et al. (2015) Scientific Reports 5: 8841; Farboud & Meyer (2015) Genetics, 199:959-971; Merkert & Martin (2016) Stem Cell Research 16(2):377-386; the disclosures of which are incorporated herein by reference in their entirety). The CRISPR system offers significant versatility in targeting of genomic modification in part because of the small size and high frequency of necessary sequence targeting elements with host genomes. CRISPR guided Cas9 nuclease requires the presence of a protospacer adjacent motif (PAM), the sequence of which depends on the bacteria species from which the Cas9 was derived (e.g. for Streptococcus pyogenes the PAM sequence is “NGG”) but such sequences are common throughout various target genomes. The PAM sequence directly downstream of the target sequence is not part of the guide RNA but is obligatory for cutting the DNA strand. However, synthetic Cas9 nucleases have been generated with novel PAM recognition, further increasing the versatility of targeting.

CRISPR related components, including Cas9 nucleases and non-Cas9 nucleases, are readily available as encoding plasmids from various sources including but not limited to those available from Addgene (Cambridge, Mass.) which may be ordered online at www(dot)addgene(dot)org.

In some instances, an employed method of target locus modification may include the use of a zinc-finger nuclease (ZFN). ZFNs consist of the sequence-independent FokI nuclease domain fused to zinc finger proteins (ZFPs). ZFPs can be altered to change their sequence specificity. Cleavage of targeted DNA requires binding of two ZFNs (designated left and right) to adjacent half-sites on opposite strands with correct orientation and spacing, thus forming a FokI dimer. The requirement for dimerization increases ZFN specificity significantly. Three or four finger ZFPs target ˜9 or 12 bases per ZFN, or ˜18 or 24 bases for the ZFN pair. ZFN pairs have been used for gene targeting at specific genomic loci in insect, plant, animal and human cells. The specificity, efficiency and versatility of targeting and replacement of homologous recombination is greatly improved through the combined use of various homology-directed repair strategies and ZFNs (see e.g., Urnov et al. (2005) Nature. 435(7042):646-5; Beumer et al (2006) Genetics. 172(4):2391-2403; Meng et al (2008) Nat Biotechnol. 26(6):695-701; Perez et al. (2008) Nat Biotechnol. 26(7):808-816; Hockemeyer et al. (2009) Nat Biotechnol. 27(9):851-7; the disclosures of which are incorporated herein by reference in their entirety).

In general, one ZFN site can be found every 125-500 bp of a random genomic sequence, depending on the assembly method. Methods for identifying appropriate ZFN targeting sites include computer-mediated methods e.g., as described in e.g., Cradick et al. (2011) BMC Bioinformatics. 12:152, the disclosure of which is incorporated herein by reference in its entirety.

ZFN related components, including ZFN nucleases, are readily available as encoding plasmids from various sources including but not limited to those available from Addgene (Cambridge, Mass.) which may be ordered online at www(dot)addgene(dot)org.

In some instances, a methods of target locus modification may include the use of a transcription activator-like effector nuclease (TALEN). Similar in principle to the ZFN nucleases, TALENs utilize the sequence-independent FokI nuclease domain fused to Transcription activator-like effectors (TALEs) proteins that, unlike ZNF, individually recognize single nucleotides. TALEs generally contain a characteristic central domain of DNA-binding tandem repeats, a nuclear localization signal, and a C-terminal transcriptional activation domain. A typical repeat is 33-35 amino acids in length and contains two hypervariable amino acid residues at positions 12 and 13, known as the “repeat variable di-residue” (RVD). An RVD is able to recognize one specific DNA base pair and sequential repeats match consecutive DNA sequences. Target DNA specificity is based on the simple code of the RVDs, which thus enables prediction of target DNA sequences. Native TALEs or engineered/modified TALEs may be used in TALENs, depending on the desired targeting.

TALENs can be designed for almost any sequence stretch. Merely the presence of a thymine at each 5′ end of the DNA recognition site is required. The specificity, efficiency and versatility of targeting and replacement of homologous recombination is greatly improved through the combined use of various homology-directed repair strategies and TALENs (see e.g., Zu et al. (2013) Nature Methods. 10:329-331; Cui et al. (2015) Scientific Reports 5:10482; Liu et al. (2012) J. Genet. Genomics. 39:209-215, Bedell et al. (2012) Nature. 491:114-118, Wang et al. (2013) Nat. Biotechnol. 31:530-532; Ding et al. (2013) Cell Stem Cell. 12:238-251; Wefers et al. (2013) Proc. Natl. Acad. Sci. U.S.A, 110:3782-3787; the disclosures of which are incorporated herein by reference in their entirety).

TALEN related components, including TALEN nucleases, are readily available as encoding plasmids from various sources including but not limited to those available from Addgene (Cambridge, Mass.) which may be ordered online at www(dot)addgene(dot)org.

Various conditions may be treated by the methods of the present disclosure, including various pain conditions, including e.g., acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, menstrual pain, combined pain conditions, and the like.

Nociceptive pain conditions may vary and may include but are not limited to e.g., those arising from central nervous system trauma, those arising from strains/sprains, those arising from burns, those arising from myocardial infarction, those arising from acute pancreatitis, post-operative pain, posttraumatic pain, renal colic, cancer pain and back pain.

Neuropathic pain conditions may vary and may include but are not limited to e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain, pain associated with chronic alcoholism, pain associated with hypothyroidism, pain associated with uremia, pain associated with multiple sclerosis, pain associated with spinal cord injury, pain associated with Parkinson's disease, epilepsy, and pain associated with vitamin deficiency.

Pain disorders, or pain associated disorders, that may be treated include but are not limited to e.g., arthritis, allodynia, a typical trigeminal neuralgia, trigeminal neuralgia, somatoform disorder, hypoesthesia, hyperalgesia, neuralgia, neuritis, neurogenic pain, analgesia, anesthesia dolorosa, causalgia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis and pain associated with cancer.

Disorders that a pain condition may be associated with include but are not limited to e.g., musculoskeletal disorders, myalgia, fibromyalgia, spondylitis, sero-negative (non-rheumatoid) arthropathies, non-articular rheumatism, dystrophinopathy, glycogenolysis, polymyositis and pyomyositis; heart disorders, vascular disorders, angina, myocardical infarction, mitral stenosis, pericarditis, Raynaud's phenomenon, scleredoma and skeletal muscle ischemia.

Pain conditions also include but are not limited to e.g., head pain, migraine, cluster headache, tension-type headache mixed headache and headache associated with vascular disorders; orofacial pain, dental pain, otic pain, burning mouth syndrome, and temporomandibular myofascial pain.

In some instances, pain conditions treatable by the herein described methods include peripheral pain conditions and pain conditions associated with peripheral nerve damage including but not limited to e.g., peripheral nerve damage, peripheral nerve damage-related conditions, conditions resulting in peripheral nerve damage or having peripheral nerve damage as a component of the pathology of the condition, and the like.

In some instances, the subject may have peripheral nerve damage. In some instances, the subject may be at elevated risk of developing peripheral nerve damage. In some instances, the pain condition may be detected using the methods described herein, including e.g., where the subject has or has not developed peripheral nerve damage. In some instances, the subject may have or be at an elevated risk of developing osteoarthritis. In some instances, the subject may have or be at an elevated risk of developing diabetic neuropathy. In some instances, the subject may have or be at an elevated risk of developing peripheral neuropathy. In some instances, the subject may have or be at an elevated risk of developing peripheral nerve damage. In some instances, the subject may have or be at an elevated risk of developing diabetes, including e.g., where the subject has or is at an elevated risk of developing diabetic neuropathy.

In some instances, the subject treated through the methods of the present disclosure may not have one or more conditions, including e.g., where the pain condition treated in the subject methods is not characterized by or does not include a spinal cord injury, or is not characterized by or does not include a central nervous system injury. In some instances, a subject treated through the methods of the present disclosure may have one or more conditions where, e.g., the pain condition treated in the subject methods is characterized by, does include, or is associated with a spinal cord injury, or is characterized by, does include or is associated with a central nervous system injury.

In some instances, the pain condition of the subject methods may include at least some level of nerve damage and/or may be caused at least in part by damage or dysfunction in at least one nerve fiber or component of the subject's nervous system. For example, in some instances, the pain condition may be caused at least in part by, as described above, damage or dysfunction in one or more peripheral neurons. In some instances, the pain condition may be caused at least in part by damage or dysfunction in one or more interneurons. In some instances, the pain condition may be caused at least in part by one or more dysfunctional neuronal circuits, including e.g., peripheral nervous system circuits and/or central nervous system circuits. In some instances, the pain condition may include one or more of the above types of damage and/or dysfunction.

As summarized above, in the subject methods of treatment, gene therapy agents may be administered in vivo to the subject. Such in vivo administration may include delivering an effective amount, in one or more doses, of the gene therapy agent to a living mammalian subject. In some instances, the method of delivery may be sufficient to deliver the gene therapy agent, including a therapeutically effective amount of the gene therapy agent in a suitable delivery composition, to the dorsal horn of the spinal cord. Such administering may be effective to cause the subject to express an effective amount of a KCC2 polypeptide, including e.g., a full-length KCC2 polypeptide or a modified KCC2 polypeptide, in dorsal horn spinal cord neurons of the mammalian subject.

For example, a schematic depiction of a cross-section of a mammalian spinal cord is provided in FIG. 1, showing dorsal horn target areas and an infusion catheter in the subarachnoid space (not to scale). Accordingly, in some embodiments, a composition that includes the gene therapy agent may be delivered via catheter in the subarachnoid space to administer a therapeutically effective amount of the gene therapy agent in a suitable delivery composition, to the dorsal horn of the spinal cord. Delivery of such composition in such a manner may be employed, in some instances, to cause the subject to express an effective amount of a desired KCC2 polypeptide in dorsal horn spinal cord neurons, thereby treating the subject for a pain condition.

Administration of compositions of the present disclosure to the spinal cord of a subject may vary, e.g., with respect to the position(s) along the spinal cord where the composition is delivered. Compositions may be delivered to essentially any desired position, or combination of positions, along the rostral/caudal axis of the spinal cord, including e.g., positions in cervical, thoracic, lumbar, sacral, and/or coccygeal regions of the spinal cord. For example, in some instances, composition may be delivered to one or more of the C1, C2, C3, C4, C5, C6, C7, C8, C9, T1, T2, T3, T4, T5, T6, T7, T8, T9, T10, T11, T12, T13, T14, T15, L1, L2, L3, L4, L5, L6, L7, S1, S2, S3, S4, or S5 positions of the spinal cord where present. In some instances, a composition may be delivered to a lumbar region of the spinal cord, including e.g., an L1, L2, L3, L4, L5, or L6 region of the lumbar spinal cord, or a combination of lumbar regions thereof, including but not limited to e.g., L3/L4, L5/L6, or L3/L4 and L5/L6. In some instances, a composition of the present disclosure may be administered to one or more positions of a mammalian spinal cord that correspond anatomically with one or more positions of another mammalian spinal cord. For example, in some instances, a composition may be administered to one or more positions in a human spinal cord corresponding to one or more of the L1, L2, L3, L4, L5, or L6 regions of the rat spinal cord.

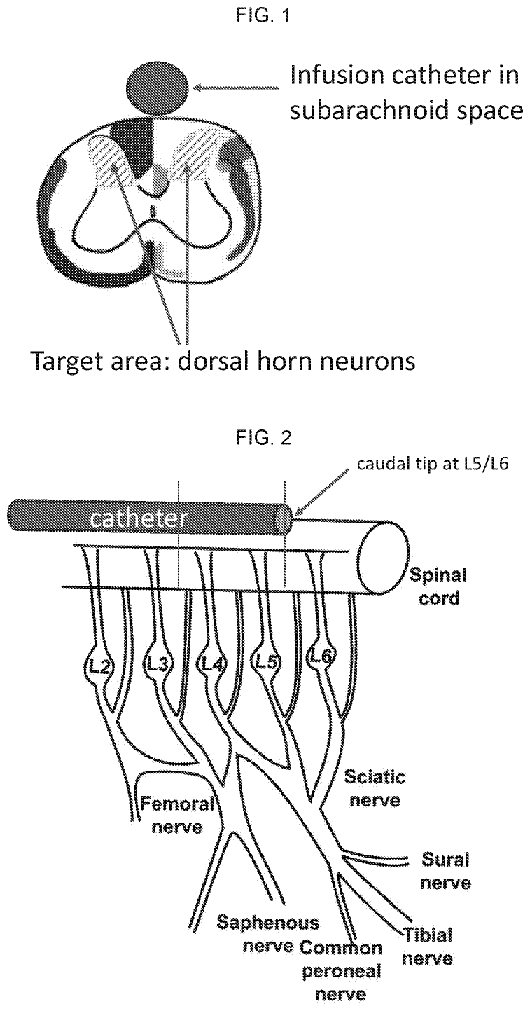

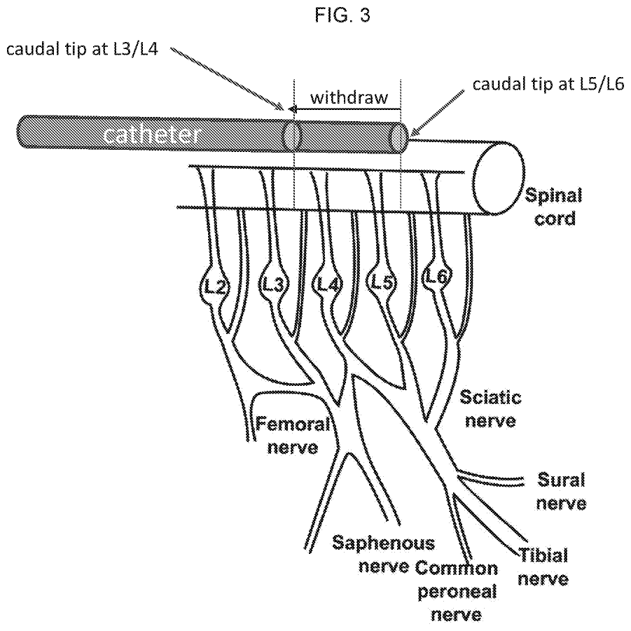

As a non-limiting example as depicted in FIG. 2, in some instances, a catheter, or other dispense device, may be inserted such that the caudal tip is present at a target location, where the target location may vary. For example, as depicted, the caudal tip of the catheter may be positioned as desired at an L5/L6 position of the rat spinal cord, or a corresponding position in another mammal, such as e.g., a corresponding position in a human subject. In some instances, the catheter may be moved during or after dispensing of the composition, e.g., to dispense to another area and/or to dispense over a wider area. During such movements, a catheter, or other dispense device, may be moved in any convenient and appropriate direction. As a non-limiting example as depicted in FIG. 3, in some instances, a catheter, or other dispense device, may be employed for a first dispense at first position (e.g., an L5/L6 position of the rat spinal cord or corresponding position in another mammal) and then withdrawn to a second position (an L3/L4 position of the rat spinal cord or corresponding position in another mammal) for a second dispense. Such delivery strategies may be readily modified or adapted as desired for dispensing to multiple different locations (including 2 or more, 3 or more, 4 or more, etc., different locations), dispensing to alternative locations (i.e., locations other than those specifically described), dispensing into corresponding locations in different species, and the like.

During and/or following delivery of one or more gene therapy vector compositions to a subject, the subject may be assessed. Assessments may be qualitative or quantitative. Such assessments may be performed for a variety of reasons, including e.g., to assess the efficacy of the administered composition, to assess whether additional dosing may be indicated and/or whether dosing should be adjusted and/or terminated, to assess whether alternative locations should be targeted, etc. Such assessments may employ various assessment methods. For example, in some instances, a behavioral assessment may be performed, including not limited to e.g., one or more of the behavioral assessments described herein.

In some instances, methods of the present disclosure may employ one or more pain assessments and/or measurements. Characteristics captured in a pain assessment may include, but are not limited to, one or more of pain intensity, pain location, pain duration and pain description.

In some instances, a pain assessment may make use of one or more pain assessments scales, including but not limited to e.g., the FLACC (Face, Legs, Activity, Cry and Consolability) assessment, the Wong-Baker faces pain scale, the Visual Analogue scale, the PQRST pain assessment method, the CRIES Scale, the COMFORT Scale, the McGill Pain Scale, the Mankoski Pain Scale, and the like. In some instances, a Behavioral pain scale (BPS) and/or a critical care pain observation tool (CPOT) can be used. In some instances, pain assessment may make use of one or more pain assessment devices, such as but not limited to e.g., probes (including e.g., electrical probes, thermal probes, etc.), monofilament devices (including e.g., those which apply a pre-determined force depending on the bend of the monofilament), and the like.

In some instances, a pain assessment may include or exclude one or more potential physiological indicators of pain, including but not limited to e.g., increased heart rate may, shift in respiratory rate and/or pattern (e.g., an increase, decrease or change pattern/rate), increase in blood pressure, decrease in oxygen saturation, and the like.

As summarized above, methods of the present disclosure include detecting the presence of a pain condition in a subject. The subject methods of detecting a pain condition may include detecting a decreased level of free KCC2 polypeptide in a sample from a subject. By “free KCC2 polypeptide” is meant KCC2 polypeptide that is not associated with a cell, including e.g., KCC2 polypeptide present in cerebrospinal fluid (CSF) that is not associated with a cell, e.g., is not present in a cellular membrane or otherwise present in or on a cell. Accordingly, in some instances, methods of the present disclosure include detecting the presence of a pain condition in a subject may include detecting a decreased level of free KCC2 polypeptide in a sample of CSF from a subject. In some instances, a CSF sample may be provided. In some instances, such methods may include obtaining the CSF sample from the subject, including e.g., where CSF is obtained by any convenient method of CSF collection, including e.g., lumbar puncture, cisternal puncture, ventricular puncture, and the like.

Generally, in the herein described methods of detecting a pain condition in a subject, the level of free KCC2 polypeptide in the sample is quantitated. In some instances, a pain condition may be detected when the quantitation reveals a decreased level of free KCC2 polypeptide. In some instances, a normal or an elevated level of free KCC2 polypeptide may be detected and such level(s) may be indicative of the absence of a pain condition.

In some instances, a detected level of free KCC2 polypeptide may be compared to a predetermined threshold, including e.g., where a pain condition is detected when the level of free KCC2 polypeptide is below the predetermined threshold.

Useful predetermined thresholds may be relative levels of free KCC2 polypeptide. Relative levels of free KCC2 polypeptide may be determined by a variety of means including e.g., determined by making a comparison of the levels of expression of free KCC2 polypeptide in two separate samples known to differ in their level of free KCC2 polypeptide. For example, a first sample known to have a normal or high level of free KCC2 polypeptide is measured and compared to a second cell population, known to have a low level of free KCC2 polypeptide and the comparison is used to determine a threshold level that may be used to categorize cells as either having a low or a high level of free KCC2 polypeptide.

In some instances, a threshold may be based on previously determined free KCC2 polypeptide levels, e.g., from previously performed control experiments or previously acquired reference expression levels. For example, free KCC2 polypeptide levels determined in previously analyzed samples may be used to determine free KCC2 polypeptide threshold levels. In some instances, free KCC2 polypeptide levels expected of cells obtained from healthy subjects may be used to determine normal free KCC2 polypeptide levels such that a free KCC2 polypeptide threshold that is representative of the normal marker range may be determined. In such instances, free KCC2 polypeptide levels outside, i.e., above or below, the normal marker range is considered to be either above or below the particular free KCC2 polypeptide threshold. In some instances, use of such previously determined free KCC2 polypeptide levels or previously determined threshold levels allows analysis of patient samples in the absence of a control or reference sample.

In some embodiments, a pain condition is detected when the evaluated level of free KCC2 polypeptide in a CSF sample is at least 5% less, including e.g., at least 10% less, at least 15% less, or at least 20% less than the level of free KCC2 polypeptide observed in a CSF sample from a healthy control subject or a reference level thereof. In some instances, a pain condition is identified as absent when the evaluated level of free KCC2 polypeptide in a SCF sample is at or above the level of free KCC2 polypeptide observed in a CSF sample from a healthy control subject or a reference level thereof.

In some instances, a measured level of free KCC2 polypeptide may be indicative of one or more characteristics of a pain condition in a subject. For example, in some instances, a decreased level of free KCC2 polypeptide may be indicative of an increased level of pain intensity, where e.g., the intensity of pain experienced by the subject may inversely correlate to the amount of free KCC2 polypeptide present in the sample.

Various methods may be employed to measure the level of free KCC2 polypeptide in an obtained sample, including but not limited to e.g., a Western blot assay, an enzyme-linked immunosorbent assay (ELISA), a mass spectrometry assay, or the like. Useful Western blot assays may vary and may include but are not limited to e.g., membrane, capillary, microcapillary, and nanocapillary assays.

In some embodiments, following an assay to detect the presence or absence of a pain condition, a subject may be treated accordingly. For example, in some instances, a method of the present disclosure may include detecting the presence of a pain condition based on a measured level of free KCC2 polypeptide in a sample from the subject and then treating the subject for the detected pain condition, including e.g., where the treatment may include administering to the subject a gene therapy vector that includes a nucleic acid sequence encoding a KCC2 polypeptide, including e.g., a full-length and/or modified KCC2 polypeptide, as described herein.

In some instances, treating a subject for a pain condition may include determining whether the subject has a pain condition by performing, or having performed, an assay on a CSF sample from the subject to detect whether the subject has a free KCC2 polypeptide level in the sample below a predetermined threshold, and then treating the subject for the detected pain condition. In some instances, such treatment may include administering to the subject a gene therapy agent that encodes an effective amount of KCC2 polypeptide or is effective to edit an endogenous KCC2 locus of the subject to encode a modified KCC2 polypeptide having enhanced activity relative to the endogenous KCC2 locus. In some instances, the CSF sample is obtained to determine if the subject has the pain condition.

In some instances, certain procedures may be performed when the pain condition is not detected by the assay, i.e., when the subject does not have a free KCC2 polypeptide level in the sample below a predetermined threshold (i.e., the level is normal or is above the predetermined threshold). In some instances, the assay may be performed, or may have been performed, and a free KCC2 polypeptide level in the sample below a predetermined threshold is not detected. In such subjects, various courses of action may be taken. For example, in some instances, further testing for a pain condition, including but not limited to e.g., one or more pain assessments described herein, may be performed. In some instances, a subject, in which a free KCC2 polypeptide level in the sample below a predetermined threshold is not detected, may be treated with one or more conventional therapies for pain, including but not limited to e.g., one or more pharmacological pain management therapies. In some instances, both further assessment and treatment with conventional pain management therapies may be employed.

As will be readily understood, the methods of treating described herein may, in some instances, be combined with one or more conventional treatments, including conventional treatments for pain, such as pharmacological treatments for pain.

In some instances, the methods of the instant disclosure may be used without any additional conventional therapy including e.g., where a method described herein is the sole method used to treat the subject. For example, in the case of a pain condition, the methods described herein may, in some instances, be the sole method used to treat the subject for a pain condition.

Gene Therapy Vectors

As summarized above, the methods of the present disclosure may include administering to a subject, in need thereof, one or more gene therapy vectors that include a nucleic acid encoding one or more KCC2 polypeptides, including e.g., full-length KCC2 polypeptides as well as modified, such as truncated and/or mutated, KCC2 polypeptides, which polypeptides are described in more detail below. Gene therapy vectors, as used herein, also include, in some instances, components for, and thus be utilized in, performing gene editing of an endogenous locus, e.g., to encode a modified KCC2 polypeptide as described herein.

Various gene therapy vectors may be employed in practicing the methods of the present disclosure. In some instances, useful viral vectors may employ, or may be derived from, retroviruses. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV)), lentivirus, and retroviral vectors derived therefrom.

Retroviral vectors and, lentiviral vectors in some instances, may be used in practicing embodiments of the present invention. Accordingly, the term “retrovirus” or “retroviral vector”, as used herein may include, but are not limited to, “lentivirus” and “lentiviral vectors” respectively.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In some instances, useful retrovirus backbones may include, but are not limited to e.g., lentiviral backbones such as, but not limited to e.g., a human immunodeficiency (HIV) lentiviral backbone, an equine infectious anemia virus (EIAV) lentiviral backbone, or the like. In some instances, useful retrovirus backbones may include, but are not limited to e.g., gammaretrovirus backbones such as, but not limited to e.g., a Moloney murine leukemia virus (MMLV) backbone, or the like.

A nucleic acid encoding a KCC2 polypeptide may in introduced into a gene therapy vector backbone such that the KCC2 polypeptide-encoding nucleic acid is operably linked to a promoter, thereby causing the KCC2 polypeptide to be expressed. Accordingly, in some instances, a viral vector backbone may include sequence encoding a single KCC2 polypeptide. In some instances, a single vector encoding a single KCC2 polypeptide may be administered to a subject in the methods described herein.

In some instances, two or more nucleic acids and/or vectors, as described herein may be administered in combination, e.g., as part of a nucleic acid and/or vector “cocktail”. As a non-limiting example, such a cocktail may include a vector encoding a first KCC2 polypeptide and a second vector encoding a second KCC2 polypeptide, where the first and second encoded KCC2 polypeptides are different. The vectors and/or encoded polypeptides of such cocktails may be individually chosen, e.g., from those vectors and/or polypeptides described herein.

In some instances, a gene therapy vector may be co-administered in combination with a second agent. Various second agents may be employed, including e.g., where the second agent is a second gene therapy vector. The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As described above, in some instances, gene therapy vectors configured for editing a target locus, e.g., to result in the expression of a modified KCC2 polypeptide from a modified endogenous locus, may include components necessary for editing the endogenous locus. Such editing vectors may include e.g., a nucleic acid encoding the modified KCC2 polypeptide, or the modified portion of a modified KCC2 polypeptide, and one or more of the additional components sufficient to facilitate editing of the endogenous locus. In some instances, components sufficient for editing may be separately provided, co-administered, or provided simultaneously. In some instances, one or more of the additional components sufficient to facilitate editing may be encoded by the vector, including but not limited to e.g., where the vector encodes a nuclease, a targeting nucleic acid (such as e.g., a guide RNA), or the like.

Accordingly, various components, in addition to nucleic acid sequence(s) encoding a desired KCC2 polypeptide, may be included in a subject gene therapy vector of the present disclosure or, in some instances, additional components may be provided separately. Useful additional components include, but are not necessarily limited to e.g., those described as employed in the above methods and those described as employed in the following compositions.

### Compositions

Aspects of the invention also include compositions, including e.g., compositions that include one or more gene therapy vectors as described herein, compositions for editing an endogenous KCC2 locus of a subject, and the like.

In some instances, such compositions may include a viral vector that includes a nucleic acid encoding a KCC2 polypeptide. Various viral vectors may be employed including e.g., retrovirus vectors, such as but are not limited to e.g., lentiviral vectors, including e.g., human immunodeficiency (HIV) lentiviral vectors, equine infectious anemia virus (EIAV) lentiviral vectors, and the like, as well as gammaretrovirus vectors such as, but not limited to e.g., Moloney murine leukemia virus (MMLV) vectors, and the like.

In some cases, a nucleic acid comprising a nucleotide sequence encoding a KCC2 polypeptide may be present in a recombinant expression vector or may be included in a recombinant expression vector. In some embodiments, a viral construct such as, e.g., a recombinant adeno-associated virus (AAV) construct, a recombinant adenoviral construct, a recombinant lentiviral construct, a recombinant retroviral construct, etc., may be employed.

Useful vectors and components thereof include, but are not limited to e.g., those available from Oxford Genetics Limited (Oxford, UK), including but not limited to e.g., those described in PCT Pub. Nos. WO/2016/189326, WO/2017/149292, WO/2017/212264, WO/2018/189535, WO/2019/058108, and WO/2019/020992; the disclosures of which are incorporated herein by reference in their entirety.

Further examples of suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector.

Compositions of the present disclosure may be prepared in a variety of configurations. For example, in some instances, compositions of the present disclosure may be formulated in unit dosage form, including e.g., where the composition is in unit dosage form in an appropriate delivery device. Such appropriate delivery devices may vary and will include e.g., those devices sufficient to facilitate delivery of the composition to a desired location via a desired route, including e.g., intrathecal, intraspinal, subpial and/intra cisterna magna administration. Useful delivery devices, systems and components thereof include but are not limited to e.g., intrathecal delivery systems, intraspinal delivery systems, subpial delivery systems, intra cisterna magna delivery systems, catheters, pumps, infusion devices, syringes, ampules, fluid bags, bottles, tubes, patches, implants, and the like.

The amount of a composition delivered to a subject may vary and may, e.g., depend upon the subject being treated, including e.g., where a larger subject, such as a human, may receive a larger volume of than that administered to a smaller subject, such as a rat. Accordingly, the volume of dose delivered to a subject, or the amount of composition present in a delivery device configured to deliver the composition to a subject, may range from 1 microliter or less to 100 milliliters or more, including but not limited to e.g., 1 μl to 100 ml, 1 μl to 75 ml, 1 μl to 50 ml, 1 μl to 25 ml, 1 μl to 20 ml, 1 μl to 15 ml, 1 μl to 10 ml, 1 μl to 5 ml, 1 μl to 4 ml, 1 μl to 3 ml, 1 μl to 2 ml, 1 μl to 1 ml, 5 μl to 100 ml, 5 μl to 75 ml, 5 μl to 50 ml, 5 μl to 25 ml, 5 μl to 20 ml, 5 μl to 15 ml, 5 μl to 10 ml, 5 μl to 5 ml, 5 μl to 4 ml, 5 μl to 3 ml, 5 μl to 2 ml, 5 μl to 1 ml, 10 μl to 100 ml, 10 μl to 75 ml, 10 μl to 50 ml, 10 μl to 25 ml, 10 μl to 20 ml, 10 μl to 15 ml, 10 μl to 10 ml, 10 μl to 5 ml, 10 μl to 4 ml, 10 μl to 3 ml, 10 μl to 2 ml, 10 μl to 1 ml, 15 μl to 100 ml, 15 μl to 75 ml, 15 μl to 50 ml, 15 μl to 25 ml, 15 μl to 20 ml, 15 μl to 15 ml, 15 μl to 10 ml, 15 μl to 5 ml, 15 μl to 4 ml, 15 μl to 3 ml, 15 μl to 2 ml, 15 μl to 1 ml, 20 μl to 100 ml, 20 μl to 75 ml, 20 μl to 50 ml, 20 μl to 25 ml, 20 μl to 20 ml, 20 μl to 15 ml, 20 μl to 10 ml, 20 μl to 5 ml, 20 μl to 4 ml, 20 μl to 3 ml, 20 μl to 2 ml, 20 μl to 1 ml, 1 μl to 500 μl, 5 μl to 500 μl, 10 μl to 500 μl, 15 μl to 500 μl, 20 μl to 500 μl, 25 μl to 500 μl, 30 μl to 500 μl, 35 μl to 500 μl, 40 μl to 500 μl, 50 μl to 500 μl, 60 μl to 500 μl, 70 μl to 500 μl, 80 μl to 500 μl, 90 μl to 500 μl, 100 μl to 500 μl, 150 μl to 500 μl, 200 μl to 500 μl, 250 μl to 500 μl, 1 μl to 250 μl, 5 μl to 250 μl, 10 μl to 250 μl, 15 μl to 250 μl, 20 μl to 250 μl, 25 μl to 250 μl, 30 μl to 250 μl, 35 μl to 250 μl, 40 μl to 250 μl, 50 μl to 250 μl, 60 μl to 250 μl, 70 μl to 250 μl, 80 μl to 250 μl, 90 μl to 250 μl, 100 μl to 250 μl, 150 μl to 250 μl, 1 μl to 100 μl, 5 μl to 100 μl, 10 μl to 100 μl, 15 μl to 100 μl, 20 μl to 100 μl, 25 μl to 100 μl, 30 μl to 100 μl, 35 μl to 100 μl, 40 μl to 100 μl, 50 μl to 100 μl, 250 μl to 1 ml, 500 μl to 1 ml, 1 ml to 2 ml, etc.

Compositions may be formulated in various ways, including but not limited to e.g., where a composition is formulated for in vivo delivery, including e.g., in vivo delivery for the treatment of pain. Compositions formulated for in vivo delivery may adhere to certain characteristics dependent at least in part on the desired mode of in vivo delivery. For example, an in vivo delivery composition will generally be sterile and may be a defined composition. In some instances, an in vivo delivery composition may include an appropriate diluent and/or other pharmacologically appropriate components. In some instances, e.g., based on the targeted delivery site, the composition may be formulated for delivery of a small volume, e.g., microliter volume, containing an effective amount of the gene therapy agent.

Concentrations of the active components, e.g., gene therapy vectors, gene editing components, and the like, of a composition will vary. With regards to viral vectors, concentrations may be expressed as infectious particles per unit volume, e.g., microliter, milliliter, etc. In some instances, the concentration of a viral vector present in a composition of the present disclosure, including ready-to-use compositions, may range from 1×10⁵ infectious particles per milliliter (particles/ml) or less to 1×10¹² particles/ml or more, including but not limited to e.g., 1×10⁵ to 1×10¹² particles/ml, 1×10⁶ to 1×10¹² particles/ml, 1×10⁷ to 1×10¹² particles/ml, 1×10⁸ to 1×10¹² particles/ml, 1×10⁹ to 1×10¹² particles/ml, 1×10¹⁰ to 1×10¹² particles/ml, 1×10¹¹ to 1×10¹² particles/ml, 1×10⁵ to 1×10¹¹ particles/ml, 1×10⁵ to 1×10¹⁰ particles/ml, 1×10⁵ to 1×10⁹ particles/ml, 1×10⁵ to 1×10⁸ particles/ml, 1×10⁵ to 1×10⁷ particles/ml, 1×10⁵ to 1×10⁶ particles/ml, 1×10⁶ to 1×10¹¹ particles/ml, 1×10⁶ to 1×10¹⁰ particles/ml, 1×10⁶ to 1×10⁹ particles/ml, 1×10⁶ to 1×10⁸ particles/ml, 1×10⁷ to 1×10¹¹ particles/ml, 1×10⁷ to 1×10¹⁰ particles/ml, 1×10⁷ to 1×10⁹ particles/ml, 1×10⁷ to 1×10⁸ particles/ml, etc.

A vector may include one or more vector specific elements. By “vector specific elements” is meant elements that are used in making, constructing, propagating, maintaining and/or assaying the vector before, during or after its construction and/or before its use in a method as described herein. Such vector specific elements include but are not limited to, e.g., vector elements necessary for the propagation, cloning and selection of the vector during its use and may include but are not limited to, e.g., an origin of replication, a multiple cloning site, a prokaryotic promoter, a phage promoter, a selectable marker (e.g., an antibiotic resistance gene, an encoded enzymatic protein, an encoded fluorescent or chromogenic protein, etc.), and the like. Any convenient vector specific elements may find use, as appropriate, in the vectors as described herein.

Such regulatory elements will vary and may include but are not limited to, e.g., a promoter, an enhancer, an intron, a polyadenylation signal, an initiation sequence (e.g., a Kozak sequence), and the like.

In certain embodiments, transcriptional control elements are operably linked, directly or indirectly to the 5′ end of a nucleic acid encoding a KCC2 polypeptide with or without intervening “spacer” nucleic acid(s). Transcriptional control elements, methods of making and/or arranging and/or modifying transcription control elements (e.g., in expression cassettes) useful in nucleic acids as described herein may, in some instances, include those described in Liu et al., Gene Therapy (2004) 11:52-60; Zheng & Baum, Methods Mol Biol. 2008, 434:205-19; Papadakis et al., Curr Gene Ther. 2004, 4(1):89-113; the disclosures of which are incorporated herein by reference in their entirety.

K-Cl Cotransporter 2 (KCC2) Polypeptides

As summarized above, gene therapy vectors may include a nucleotide sequence encoding for a KCC2 polypeptide, including e.g., a nucleotide sequence encoding a full-length KCC2 polypeptide. KCC2 (also referred to as solute carrier family 12 member 5, electroneutral potassium-chloride cotransporter 2, K-Cl cotransporter 2, and Neuronal K-Cl cotransporter) is encoded by the SLC12A5 gene in humans (HGNC:13818) located at 20q13.12. The KCC2 protein mediates electroneutral potassium-chloride cotransport in mature neurons and is involved in neuronal chloride homeostasis as well as the regulation of dendritic spine formation and maturation.

Useful full-length KCC2 polypeptides that may be encoded by such nucleic acid sequences include e.g., mammalian full-length KCC2 polypeptides, including e.g., rat full-length KCC2 (UniProtKB Q63633; RefSeq NP_599190.1, NM_134363.1), mouse full-length KCC2 (UniProtKB Q91V14; RefSeq NP_065066.2, NM_020333.2), and human full-length KCC2 (UniProtKB Q9H2X9; NP_001128243.1, NM_001134771.1, NP_065759.1, NM_020708.4) having the following amino acid sequence:

NP_001128243.1 (isoform 1)
(SEQ ID NO: 01)
MSRRFTVTSLPPAGPARSPDPESRRHSVADPRHLPGEDVKGDGNPKESSP
FINSTDTEKGKEYDGKNMALFEEEMDTSPMVSSLLSGLANYTNLPQGSRE
HEEAENNEGGKKKPVQAPRMGTFMGVYLPCLQNIFGVILFLRLTWVVGIA
GIMESFCMVFICCSCTMLTAISMSAIATNGVVPAGGSYYMISRSLGPEFG
GAVGLCFYLGTTFAGAMYILGTIEILLAYLFPAMAIFKAEDASGEAAAML
NNMRVYGTCVLTCMATVVFVGVKYVNKFALVFLGCVILSILAIYAGVIKS
AFDPPNFPICLLGNRTLSRHGFDVCAKLAWEGNETVTTRLWGLFCSSRFL
NATCDEYFTRNNVTEIQGIPGAASGLIKENLWSSYLTKGVIVERSGMTSV
GLADGTPIDMDHPYVFSDMTSYFTLLVGIYFPSVTGIMAGSNRSGDLRDA
QKSIPTGTILAIATTSAVYISSVVLFGACIEGVVLRDKFGEAVNGNLVVG
TLAWPSPWVIVIGSFFSTCGAGLQSLTGAPRLLQAISRDGIVPFLQVFGH
GKANGEPTWALLLTACICEIGILIASLDEVAPILSMFFLMCYMFVNLACA
VQTLLRTPNWRPRFRYYHWTLSFLGMSLCLALMFICSWYYALVAMLIAGL
IYKYIEYRGAEKEWGDGIRGLSLSAARYALLRLEEGPPHTKNWRPQLLVL
VRVDQDQNVVHPQLLSLTSQLKAGKGLTIVGSVLEGTFLENHPQAQRAEE
SIRRLMEAEKVKGFCQVVISSNLRDGVSHLIQSGGLGGLQHNTVLVGWPR
NWRQKEDHQTWRNFIELVRETTAGHLALLVTKNVSMFPGNPERFSEGSID
VWWIVHDGGMLMLLPFLLRHHKVWRKCKMRIFTVAQMDDNSIQMKKDLTT
FLYHLRITAEVEVVEMHESDISAYTYEKTLVMEQRSQILKQMHLTKNERE
REIQSITDESRGSIRRKNPANTRLRLNVPEETAGDSEEKPEEEVQLIHDQ
SAPSCPSSSPSPGEEPEGEGETDPEKVHLTWTKDKSVAEKNKGPSPVSSE
GIKDFFSMKPEWENLNQSNVRRMHTAVRLNEVIVKKSRDAKLVLLNMPGP
PRNRNGDENYMEFLEVLTEHLDRVMLVRGGGREVITIYS.
NP_065759.1 (isoform 2)
(SEQ ID NO: 02)
MLNNLTDCEDGDGGANPGDGNPKESSPFINSTDTEKGKEYDGKNMALFEE
EMDTSPMVSSLLSGLANYTNLPQGSREHEEAENNEGGKKKPVQAPRMGTF
MGVYLPCLQNIFGVILFLRLTWVVGIAGIMESFCMVFICCSCTMLTAISM
SAIATNGVVPAGGSYYMISRSLGPEFGGAVGLCFYLGTTFAGAMYILGTI
EILLAYLFPAMAIFKAEDASGEAAAMLNNMRVYGTCVLTCMATVVFVGVK
YVNKFALVFLGCVILSILAIYAGVIKSAFDPPNFPICLLGNRTLSRHGFD
VCAKLAWEGNETVTTRLWGLFCSSRFLNATCDEYFTRNNVTEIQGIPGAA
SGLIKENLWSSYLTKGVIVERSGMTSVGLADGTPIDMDHPYVFSDMTSYF
TLLVGIYFPSVTGIMAGSNRSGDLRDAQKSIPTGTILAIATTSAVYISSV
VLFGACIEGVVLRDKFGEAVNGNLVVGTLAWPSPWVIVIGSFFSTCGAGL
QSLTGAPRLLQAISRDGIVPFLQVFGHGKANGEPTWALLLTACICEIGIL
IASLDEVAPILSMFFLMCYMFVNLACAVQTLLRTPNWRPRFRYYHWTLSF
LGMSLCLALMFICSWYYALVAMLIAGLIYKYIEYRGAEKEWGDGIRGLSL
SAARYALLRLEEGPPHTKNWRPQLLVLVRVDQDQNVVHPQLLSLTSQLKA
GKGLTIVGSVLEGTFLENHPQAQRAEESIRRLMEAEKVKGFCQVVISSNL
RDGVSHLIQSGGLGGLQHNTVLVGWPRNWRQKEDHQTWRNFIELVRETTA
GHLALLVTKNVSMFPGNPERFSEGSIDVWWIVHDGGMLMLLPFLLRHHKV
WRKCKMRIFTVAQMDDNSIQMKKDLTTFLYHLRITAEVEVVEMHESDISA
YTYEKTLVMEQRSQILKQMHLTKNEREREIQSITDESRGSIRRKNPANTR
LRLNVPEETAGDSEEKPEEEVQLIHDQSAPSCPSSSPSPGEEPEGEGETD
PEKVHLTWTKDKSVAEKNKGPSPVSSEGIKDFFSMKPEWENLNQSNVRRM
HTAVRLNEVIVKKSRDAKLVLLNMPGPPRNRNGDENYMEFLEVLTEHLDR
VMLVRGGGREVITIYS

Mammalian orthologs of human KCC2 include but are not limited to e.g., Bos taurus (NP_001193309.1, NM_001206380.1), Equus caballus (UniProtKB F7DY57), Canis familiaris (UniProtKB F1PGY7), Felis catus (UniProtKB A0A212URB1), as well as non-human primate orthologs such as e.g., Macaca mulatta (XP_001104798.1, XM_001104798.3; UniProtKB A0A1 D5Q4H1) and Pan troglodytes (XP_016793510.1, XM_016938021.1; UniProtKB A0A213SRL5).

As summarized above, gene therapy vectors may include a nucleotide sequence encoding for a modified KCC2 polypeptide, including e.g., a nucleotide sequence encoding a truncated KCC2 polypeptide, a nucleotide sequence encoding a KCC2 polypeptide having one or more substitution mutations, and a nucleotide sequence encoding a truncated KCC2 polypeptide having one or more substitution mutations.

Useful modified KCC2 polypeptides that may be encoded by such nucleic acid sequences include e.g., a KCC2 polypeptide, e.g., the human full-length KCC2 amino acid sequence provided above, modified by one or more of truncation, amino acid substitution, or the like.

In some instances, a modified KCC2 polypeptide encoded in a vector of the present disclosure will have, or an endogenous locus may be modified to encode, a polypeptide having at least 80% sequence identity with one or more of the above provided full-length KCC2 amino acid sequences, including but not limited to e.g., a polypeptide with at least 85%, at least 90%, at least 91%, at least 92%, at least 93%, at least 94%, at least 95%, at least 96%, at least 97%, at least 98%, or at least 99% sequence identity with one or more of the above provided full-length KCC2 amino acid sequences. In general, modified KCC2 polypeptides encoded in a vector of the present disclosure, or a locus modified to encode a modified KCC2 polypeptide, will share less than 100% sequence identity with the above provided full-length KCC2 amino acid sequences.

Truncated modified KCC2 polypeptides may include N-terminal or C-terminal truncations. In some instances, a truncated KCC2 polypeptide may include an N-terminal truncation relative to a wild-type KCC2 polypeptide (such as e.g., one or more of the full-length KCC2 polypeptides described above) of one or more amino acid residues, such as e.g., two or more, three or more, four or more, five or more, etc.

The degree of truncation of modified KCC2 polypeptides may vary and may range from 1 amino acid residue to 50 amino acid residues or more, including but not limited to e.g., 1 to 50 aa residues, 5 to 50 aa residues, 10 to 50 aa residues, 15 to 50 aa residues, 20 to 50 aa residues, 25 to 50 aa residues, 30 to 50 aa residues, 35 to 50 aa residues, 40 to 50 aa residues, 5 to 45 aa residues, 5 to 40 aa residues, 5 to 35 aa residues, 5 to 30 aa residues, 5 to 25 aa residues, 5 to 20 aa residues, 5 to 15 aa residues, 5 to 10 aa residues, 10 to 45 aa residues, 10 to 40 aa residues, 10 to 35 aa residues, 10 to 30 aa residues, 10 to 25 aa residues, 10 to 20 aa residues, 10 to 15 aa residues, 10 to 10 aa residues, 20 to 40 aa residues, 30 to 40 aa residues, 20 to 30 aa residues, etc.

In some instances, an N-terminal truncation of a KCC2 polypeptide may include at least a 5 amino acid residue truncation relative to the wild-type counterpart, including but not limited to e.g., at least a 10 amino acid residue truncation, at least a 15 amino acid residue truncation, at least a 20 amino acid residue truncation, at least a 25 amino acid residue truncation, at least a 30 amino acid residue truncation, at least a 35 amino acid residue truncation, or at least a 40 amino acid residue truncation.

Useful truncated KCC2 polypeptides include e.g., the truncated human KCC2 polypeptide, and variants thereof, represented by the following amino acid sequence:

(SEQ ID NO: 03)
MGDGNPKESSPFINSTDTEKGKEYDGKNMALFEEEMDTSPMVSSLLSGLA
NYTNLPQGSREHEEAENNEGGKKKPVQAPRMGTFMGVYLPCLQNIFGVIL
FLRLTWVVGIAGIMESFCMVFICCSCTMLTAISMSAIATNGVVPAGGSYY
MISRSLGPEFGGAVGLCFYLGTTFAGAMYILGTIEILLAYLFPAMAIFKA
EDASGEAAAMLNNMRVYGTCVLTCMATVVFVGVKYVNKFALVFLGCVILS
ILAIYAGVIKSAFDPPNFPICLLGNRTLSRHGFDVCAKLAWEGNETVTTR
LWGLFCSSRFLNATCDEYFTRNNVTEIQGIPGAASGLIKENLWSSYLTKG
VIVERSGMTSVGLADGTPIDMDHPYVFSDMTSYFTLLVGIYFPSVTGIMA
GSNRSGDLRDAQKSIPTGTILAIATTSAVYISSVVLFGACIEGVVLRDKF
GEAVNGNLVVGTLAWPSPWVIVIGSFFSTCGAGLQSLTGAPRLLQAISRD
GIVPFLQVFGHGKANGEPTWALLLTACICEIGILIASLDEVAPILSMFFL
MCYMFVNLACAVQTLLRTPNWRPRFRYYHWTLSFLGMSLCLALMFICSWY
YALVAMLIAGLIYKYIEYRGAEKEWGDGIRGLSLSAARYALLRLEEGPPH
TKNWRPQLLVLVRVDQDQNVVHPQLLSLTSQLKAGKGLTIVGSVLEGTFL
ENHPQAQRAEESIRRLMEAEKVKGFCQVVISSNLRDGVSHLIQSGGLGGL
QHNTVLVGWPRNWRQKEDHQTWRNFIELVRETTAGHLALLVTKNVSMFPG
NPERFSEGSIDVWWIVHDGGMLMLLPFLLRHHKVWRKCKMRIFTVAQMDD
NSIQMKKDLTTFLYHLRITAEVEVVEMHESDISAYTYEKTLVMEQRSQIL
KQMHLTKNEREREIQSITDESRGSIRRKNPANTRLRLNVPEETAGDSEEK
PEEEVQLIHDQSAPSCPSSSPSPGEEPEGEGETDPEKVHLTWTKDKSVAE
KNKGPSPVSSEGIKDFFSMKPEWENLNQSNVRRMHTAVRLNEVIVKKSRD
AKLVLLNMPGPPRNRNGDENYMEFLEVLTEHLDRVMLVRGGGREVITIYS.

In some instances, a modified KCC2 polypeptide encoded in a vector of the present disclosure will have, or an endogenous locus may be modified to encode, a polypeptide having at least 80% sequence identity with the above provided amino acid sequence, including but not limited to e.g., a polypeptide with at least 85%, at least 90%, at least 91%, at least 92%, at least 93%, at least 94%, at least 95%, at least 96%, at least 97%, at least 98%, or at least 99% sequence identity with the above provided amino acid sequence. In some instances, a modified KCC2 polypeptide encoded in a vector of the present disclosure will have, or an endogenous locus may be modified to encode, a polypeptide having 100% sequence identity with the above provided amino acid sequence.

Mutated modified KCC2 polypeptides may include one or more amino acid substitutions, where such substitutions may include conservative substitutions, non-conservative substitutions, or combinations thereof. Mutated KCC2 polypeptide amino acid substitutions may be identified relative to a wild-type KCC2 polypeptide (such as e.g., one or more of the full-length KCC2 polypeptides described above). The number of substitutions in a modified KCC2 polypeptide may vary and may range from 1 to 10 or more, including but not limited to e.g., 1 or more, 2 or more, 3 or more, 4 or more, 5 or more, 6 or more, 7 or more, 8 or more, 9 or more, 10 or more, etc. In some instances, one or more of the substitutions present in a modified KCC2 polypeptide may be an alanine substitution, such as but not limited to e.g., a T to A substitution, or the like. In some instances, one or more of the substitutions present in a modified KCC2 polypeptide may be an aspartic acid substitution, such as but not limited to e.g., a T to D substitution, a S to D substitution, or the like.

In some instances, one or more of the substitutions present in a modified KCC2 polypeptide may be, relative to the full-length human KCC2 amino acid sequence provided above or one or more relative positions from a related ortholog, a 1906A substitution, a T934D substitution, a S937D substitution, or a T1007A substitution. In some instances, a modified KCC2 polypeptide of the present disclosure may include a combination of substitutions that includes one or more of, or all of, a 1906A substitution, a T934D substitution, a S937D substitution, and a T1007A substitution.

Useful modified KCC2 polypeptides include but are not limited to e.g., a truncated KCC2 polypeptide, a KCC2 polypeptide having a 1906A substitution, a KCC2 polypeptide having a T934D substitution, a KCC2 polypeptide having a S937D substitution, a KCC2 polypeptide having a T1007A substitution, a truncated KCC2 polypeptide having a 1906A substitution, a truncated KCC2 polypeptide having a T934D substitution, a truncated KCC2 polypeptide having a S937D substitution, a truncated KCC2 polypeptide having a T1007A substitution, a KCC2 polypeptide having some combination of substitutions selected from 1906A, T934D, S937D and T1007A, a truncated KCC2 polypeptide having some combination of substitutions selected from 1906A, T934D, S937D and T1007A, and the like.

In some instances, a modified KCC2 polypeptide encoded in a vector of the present disclosure will have, or an endogenous locus may be modified to encode, a polypeptide having an N-terminal truncation relative to a wild-type KCC2 polypeptide and one or more substitution mutations relative to the wild-type KCC2 polypeptide.

Useful modified KCC2 polypeptides include but are not limited to e.g., the modified KCC2 polypeptides represented by the following amino acid sequences, and variants thereof:

(SEQ ID NO: 04)
MGDGNPKESSPFINSTDTEKGKEYDGKNMALFEEEMDTSPMVSSLLSGLA
NYTNLPQGSREHEEAENNEGGKKKPVQAPRMGTFMGVYLPCLQNIFGVIL
FLRLTWVVGIAGIMESFCMVFICCSCTMLTAISMSAIATNGVVPAGGSYY
MISRSLGPEFGGAVGLCFYLGTTFAGAMYILGTIEILLAYLFPAMAIFKA
EDASGEAAAMLNNMRVYGTCVLTCMATVVFVGVKYVNKFALVFLGCVILS
ILAIYAGVIKSAFDPPNFPICLLGNRTLSRHGFDVCAKLAWEGNETVTTR
LWGLFCSSRFLNATCDEYFTRNNVTEIQGIPGAASGLIKENLWSSYLTKG
VIVERSGMTSVGLADGTPIDMDHPYVFSDMTSYFTLLVGIYFPSVTGIMA
GSNRSGDLRDAQKSIPTGTILAIATTSAVYISSVVLFGACIEGVVLRDKF
GEAVNGNLVVGTLAWPSPWVIVIGSFFSTCGAGLQSLTGAPRLLQAISRD
GIVPFLQVFGHGKANGEPTWALLLTACICEIGILIASLDEVAPILSMFFL
MCYMFVNLACAVQTLLRTPNWRPRFRYYHWTLSFLGMSLCLALMFICSWY
YALVAMLIAGLIYKYIEYRGAEKEWGDGIRGLSLSAARYALLRLEEGPPH
TKNWRPQLLVLVRVDQDQNVVHPQLLSLTSQLKAGKGLTIVGSVLEGTFL
ENHPQAQRAEESIRRLMEAEKVKGFCQVVISSNLRDGVSHLIQSGGLGGL
QHNTVLVGWPRNWRQKEDHQTWRNFIELVRETTAGHLALLVTKNVSMFPG
NPERFSEGSIDVWWIVHDGGMLMLLPFLLRHHKVWRKCKMRIFTVAQMDD
NSIQMKKDLTTFLYHLRITAEVEVVEMHESDISAYTYEKALVMEQRSQIL
KQMHLTKNEREREIQSIDDEDRGSIRRKNPANTRLRLNVPEETAGDSEEK
PEEEVQLIHDQSAPSCPSSSPSPGEEPEGEGETDPEKVHLAWTKDKSVAE
KNKGPSPVSSEGIKDFFSMKPEWENLNQSNVRRMHTAVRLNEVIVKKSRD
AKLVLLNMPGPPRNRNGDENYMEFLEVLTEHLDRVMLVRGGGREVITIYS;
and
(SEQ ID NO: 05)
MLNNLTDCEDGDGGANPGDGNPKESSPFINSTDTEKGKEYDGKNMALFEE
EMDTSPMVSSLLSGLANYTNLPQGSREHEEAENNEGGKKKPVQAPRMGTF
MGVYLPCLQNIFGVILFLRLTWVVGIAGIMESFCMVFICCSCTMLTAISM
SAIATNGVVPAGGSYYMISRSLGPEFGGAVGLCFYLGTTFAGAMYILGTI
EILLAYLFPAMAIFKAEDASGEAAAMLNNMRVYGTCVLTCMATVVFVGVK
YVNKFALVFLGCVILSILAIYAGVIKSAFDPPNFPICLLGNRTLSRHGFD
VCAKLAWEGNETVTTRLWGLFCSSRFLNATCDEYFTRNNVTEIQGIPGAA
SGLIKENLWSSYLTKGVIVERSGMTSVGLADGTPIDMDHPYVFSDMTSYF
TLLVGIYFPSVTGIMAGSNRSGDLRDAQKSIPTGTILAIATTSAVYISSV
VLFGACIEGVVLRDKFGEAVNGNLVVGTLAWPSPWVIVIGSFFSTCGAGL
QSLTGAPRLLQAISRDGIVPFLQVFGHGKANGEPTWALLLTACICEIGIL
IASLDEVAPILSMFFLMCYMFVNLACAVQTLLRTPNWRPRFRYYHWTLSF
LGMSLCLALMFICSWYYALVAMLIAGLIYKYIEYRGAEKEWGDGIRGLSL
SAARYALLRLEEGPPHTKNWRPQLLVLVRVDQDQNVVHPQLLSLTSQLKA
GKGLTIVGSVLEGTFLENHPQAQRAEESIRRLMEAEKVKGFCQVVISSNL
RDGVSHLIQSGGLGGLQHNTVLVGWPRNWRQKEDHQTWRNFIELVRETTA
GHLALLVTKNVSMFPGNPERFSEGSIDVWWIVHDGGMLMLLPFLLRHHKV
WRKCKMRIFTVAQMDDNSIQMKKDLTTFLYHLRITAEVEVVEMHESDISA
YTYEKALVMEQRSQILKQMHLTKNEREREIQSIDDEDRGSIRRKNPANTR
LRLNVPEETAGDSEEKPEEEVQLIHDQSAPSCPSSSPSPGEEPEGEGETD
PEKVHLAWTKDKSVAEKNKGPSPVSSEGIKDFFSMKPEWENLNQSNVRRM
HTAVRLNEVIVKKSRDAKLVLLNMPGPPRNRNGDENYMEFLEVLTEHLDR
VMLVRGGGREVITIYS.

In some instances, a modified KCC2 polypeptide encoded in a vector of the present disclosure will have, or an endogenous locus may be modified to encode, a polypeptide having at least 80% sequence identity with the above provided amino acid sequence, including but not limited to e.g., a polypeptide with at least 85%, at least 90%, at least 91%, at least 92%, at least 93%, at least 94%, at least 95%, at least 96%, at least 97%, at least 98%, or at least 99% sequence identity with the above provided amino acid sequence. In some instances, a modified KCC2 polypeptide encoded in a vector of the present disclosure will have, or an endogenous locus may be modified to encode, a polypeptide having 100% sequence identity with the above provided amino acid sequence.

Modifications of KCC2 polypeptides are not necessarily limited to truncations and substitutions. Accordingly, in some instances, a modified KCC2 polypeptide may include a truncation, a substitution, or a combination thereof and may be further modified. In some instances, relative to the corresponding wild-type sequence, a modified KCC2 polypeptide may not include further modifications besides a N-terminal truncation and/or a substitution, including but not limited to e.g., the N-terminal truncations and/or substitutions identified above.

Modified polypeptides may also include, in some instances, those polypeptides that have been modified to improve their use as a therapeutic. Such polypeptide modification may include modification to achieve the minimal active sequence (MAS), deletion of one or more consecutive amino acid(s) to achieve the MAS, combinatorial deletion with two or more positions omitted independently to achieve the MAS, structure simplification (e.g., following alanine or D amino acid scanning to identify non-active sites), cleavage site elimination, modification to reduce hydrogen bonding, modification to increase membrane permeability (e.g., by modifying the overall or regional (e.g., surface) charge of a polypeptide), and the like. Polypeptide modifications have been described, e.g., by Vlieghe et al. (2010) Drug Discovery Today. 15:(1/2) 40-56, the disclosure of which is incorporated herein by reference.

The ordinary skilled artisan will readily understand where a polypeptide modification may be encoded (e.g., an amino acid substitution, amino acid addition, amino acid truncation, etc.) in a nucleic acid. The ordinary skilled artisan will also readily understand that where a polypeptide modification is initially synthetically produced (e.g., through enzymatic truncation of a polypeptide) such modification may, in some instances, also be achieved by modifying a nucleic acid that encodes the polypeptide (e.g., by truncating the nucleic acid).

Nucleic Acids

As summarized above, the compositions of the present disclosure, and those employed in the instant methods, include nucleic acid components. For example, gene therapy vector compositions may include vector backbone nucleic acids, KCC2 polypeptide-encoding nucleic acids, nucleic acid components of gene editing systems, and the like. Accordingly, any polypeptide described herein may be provided by an appropriate nucleic acid encoding the polypeptide. As such, nucleic acids of the present disclosure, and the subcomponents (i.e., domains, elements, sequences, etc.) thereof, will vary.

Depending on the host/vector system utilized, any of a number of suitable transcription and translation control elements, including constitutive and inducible promoters, transcription enhancer elements, transcription terminators, etc. may be used in an expression vector (see e.g., Bitter et al. (1987) Methods in Enzymology, 153:516-544). Such elements may be operably linked to a nucleic acid encoding a desired polypeptide.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., during an organismal response to pain).

Suitable promoters include but are not limited to cytomegalovirus (CMV) promoters, β-actin promoters (ACTB), elongation factor-1α (EF1α) promoters, phosphoglycerate kinase (PGK) promoters, ubiquitinC (UbC) promoters, and the like. In some instances, suitable promoters may also include promoters active in neuronal cell types, such as but not limited to e.g., synapsin promoters, such as but not limited to e.g., the human synapsin 1 (hSYN1) gene promoter (see, e.g., GenBank HUMSYNIB, M55301) and analogs thereof.

Other examples of promoter sequences operable in a neuron include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a thy-1 promoter (see, e.g., Chen et al. (1987) Cell 51:7-19); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; and a CMV enhancer/platelet-derived growth factor-β promoter (see, e.g., Liu et al. (2004) Gene Therapy 11:52-60). Various promoters operable in a neuron, including neuron-specific promoters and other control elements (e.g., enhancers), may be employed in some instances.

Additional Composition Components

Compositions of the present disclosure may further include one or more additional components, e.g., in addition to nucleic acids and/or in additional to one or more gene therapy vectors as described herein. Such additional components may vary

In some instances, a composition that includes one or more gene therapy vector components includes an appropriate diluent, e.g., a suitable solution or liquid for dissolving or suspending a vector as described herein. Such diluents may vary and may depend upon, e.g., the concentration of vector to be suspended, the pharmaceutical formulation of the vector, the mode of delivery of the vector, the method of storage of the vector, and the like. In some instances, a suitable solution or liquid may include but is not limited to, e.g., aqueous solutions, water (e.g., nuclease-free water, water for injection (WFI), etc.), saline, phosphate buffered saline (PBS), tris buffer saline (TBS), tris-EDTA (TE) buffer, combinations thereof, and the like. Pharmaceutical formulations of vectors are discussed in more detail below.

A pharmaceutical composition of the instant disclosure is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for infusion and/or injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Kolliphor EL or phosphate buffered saline (PBS). In all cases, the composition is generally sterile and should be fluid to the extent that easy syringeability or infusibility exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is advantageous, in some instances, to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure are dictated by and directly dependent on the unique characteristics of the active compound and the particular therapeutic effect to be achieved, and the limitations inherent in the art of compounding such an active compound for the treatment of individuals.

### Kits

Also provided are kits for use in the subject methods. The subject kits may include any combination of components and compositions for performing the subject methods. In some embodiments, a kit can include the following: a vector, a vector delivery device, a suitable buffer and any combination thereof.

In some embodiments, a subject kit includes vector and a suitable diluent for resuspending and/or diluting the vector before use where the vector and the diluent are present in separate containers. In some instances, a subject kit may include one or more pre-formulated doses of vector in “ready-to-use” format (e.g., as injectable vector, infusible vector, etc.). In instances where a dosing regimen is desired that includes multiple administrations of one or more vectors, a subject kit may include two or more doses of vector, in a pre-formulated or an unformulated configuration, and may, optionally, include instructions (e.g., instructions as to when each dose should be administered, instruction for preparing unformulated doses, instructions for dose delivery, etc.).

In some instances, a subject kit may include one or more testing reagents or testing devices or combinations thereof for assaying a subject's need for therapy (e.g., before or after therapy), assaying the effectiveness of therapy (e.g., during or after therapy), etc. Such devices may include but are not limited to, e.g., one or more CSF collection devices, one or more components of a CSF collection system, such as e.g., a CSF collection container. In some instances, a subject kit may include one or more components for assaying a subject for a pain assay, or collecting a sample to be used in such an assay, and one or more treatment components (including e.g., one or more gene therapy vectors) for treating the subject based on the outcome of the assay.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Spinal Nerve Ligation (SNL) Model of Neuropathic Pain in Rats

This study demonstrates the effectiveness of agents of the present disclosure in a rat model of neuropathic pain, induced by Spinal Nerve Ligation (SNL). The Chung SPL model is a model for neuropathic pain that enables the measurement of the animal's pain threshold immediately after the animal awakes from the surgery. While under anesthesia using medetomine/ketamine sodium and after the area is shaved, the rat is placed in a prone position and the left paraspinal muscles are separated from the spinous process at the L4-S2 levels. The L6 vertebral transverse process is carefully removed with a small rongeur to visually identify the L5-L6 spinal nerves. A tight knot is performed (ligation) surrounding the left L5+L6 spinal nerves using 6-0 silk thread. The muscle is then closed with 3-0 silk sutures and the skin is closed by a clamp. Following surgery, the rats are returned to the cage and remained under a heating lamp until they awake.

The agents tested in this example included: (1) lentivirus vector containing cytomegalovirus (CMV) promoter-driven full-length human KCC2 (CMV-FL hKCC2); (2) lentivirus vector containing human synapsin promoter-driven full-length human KCC2 (hSyn1-FL hKCC2); (3) lentivirus vector containing CMV promoter-driven truncated human KCC2 (CMV-trunc hKCC2); (4) lentivirus vector containing CMV promoter-driven mutant human KCC2 (CMV-mut hKCC2), and appropriate controls.

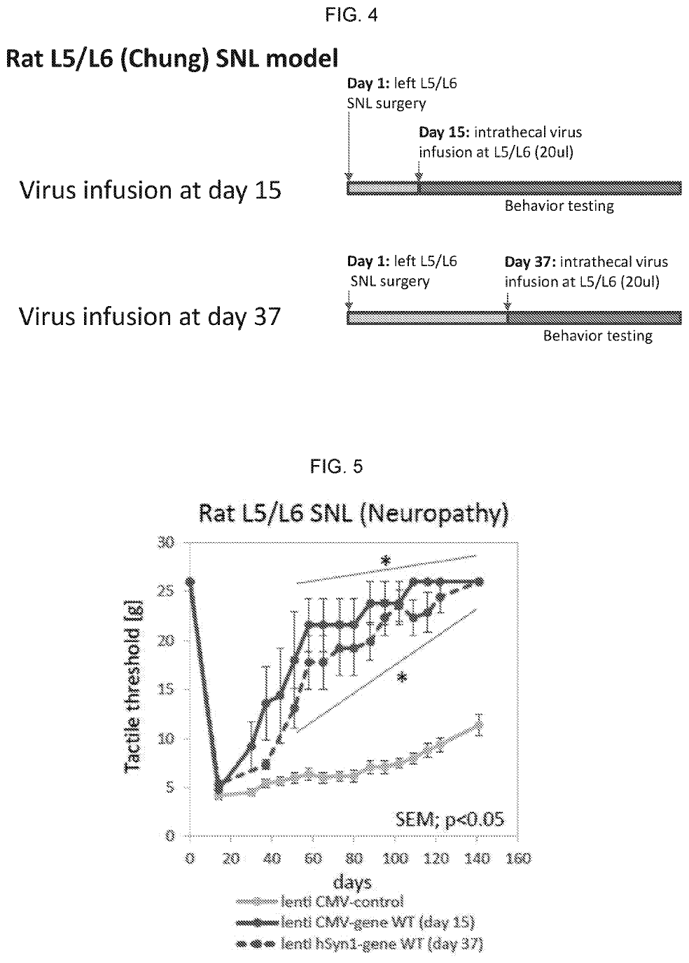

Agents were stored ready-to-use at −80° C., gently thawed, aliquoted and kept on ice prior to administration. Agents were delivered via intrathecal catheter at 20 μl per rat. After a small incision at back of neck and small opening in the atlanto-occipital membrane of the cisterna magna and dura, PE-10 catheter was inserted until L5+L6 spinal nerves and 20 μl of the agent compositions were injected. Then the catheter was slowly withdrawn after 3 minutes of incubation. FIG. 4 provides exemplary experimental timelines employed in the rat SNL model study.

Animals were evaluated using various tests performed before and/or after agent or control administration, such tests included e.g., Von Frey tests, Hargreaves tests, and Noxious Pressure tests. Additional measurements were assessed during the study, including e.g., body weight measurement. Exemplary body weight measurements included measurement before SNL surgery for baseline values and once a week thereafter. Throughout the study, general clinical signs and observation were performed and recorded. Observations included changes in skin, fur, eyes, mucous membranes, occurrence of secretions and excretions (e.g. diarrhea) and autonomic activity (e.g., lacrimation, salivation, piloerection, pupil size, unusual respiratory pattern). Changes in gait, posture and response to handling, as well as the presence of abnormal behavior, tremors, convulsions, sleep and coma.

Von Frey testing provided for an evaluation of mechanical allodynia. Allodynic response to tactile stimulation was assessed using the Von Frey apparatus according to the up-down method. The rat was placed in an enclosure and positioned on a metal mesh surface, but allowed to move freely. The test began after a cessation of exploratory behavior. The set of Von Frey monofilaments provided an approximate logarithmic scale of actual force and a linear scale of perceived intensity. The operating principle of the test involves the tip of a fiber of given length and diameter pressed against the skin at right angles, where the force of application increases as long as the researcher continues to advance the probe until the fiber bends. After the fiber bends, the probe continues to advance, causing the fiber to bend more, but without additional force being applied.

Fiber sizes and the corresponding force applied are provided in the following table:

| | | | | | | | | | | | | | | | | | |
|---|---|---|---|---|---|---|---|---|---|---|---|---|---|---|---|---|---|
| Size | 1.65 | 2.36 | 2.44 | 2.83 | 3.22 | 3.61 | 3.84 | 4.08 | 4.17 | 4.31 | 4.56 | 4.74 | 4.93 | 5.07 | 5.18 | 5.46 | 5.88 |
| Force (g) | 0.008 | 0.02 | 0.04 | 0.07 | 0.16 | 0.4 | 60 | 1 | 1.4 | 2 | 4 | 6 | 8 | 10 | 15 | 26 | 60 |

Rodents exhibit a paw withdrawal reflex when the paw is unexpectedly touched. A sensory evaluator can be used on the plantar surfaces of the foot. The animal indicates sensation by pulling back its paw. The minimal force needed to elevate the withdrawal reflex is considered/designated as the value of reference. Decreases in force needed to induce withdrawal are indicative of allodynia, as the force applied is a non-painful stimulus under normal conditions.

The Noxious Pressure test provided for a measurement of hyperalgesia. Noxious Pressure was measured using Ugo Basile Pressure Application Measurement. A paw pressure applicator was used to generate an increasing force. When the animal displayed pain by either withdrawing its paw or vocalizing, the pressure was automatically paused. A maximum of 400 g (0 to 1500 g range) was used as a cutoff to avoid potential tissue injury to the animals.

Heat stimulation via the Hargreaves method provided a measurement for hyperalgesia. Thermal hyperalgesia was tested using plantar test 37370 apparatus (UGO BASILE). Each rat was placed within a plastic box (W100×L200×H145 mm) atop a glass floor. A light beam under the floor was aimed at the plantar surface of the left hind paw. Once the light beam was triggered, a timer was started. The rising temperature on the surface causes the animal to move its foot. This stops the timer. Latency to move the foot was recorded in seconds. The intensity of the light was adjusted with latency of normal paw at approximately 10 seconds and a cut-off latency of 30 seconds. The withdrawal latency for each animal was defined as the heat pain threshold.

At study termination lumbar spinal cords were collected from each animal for further analysis, including but not limited to e.g., immunohistochemistry for various markers such as e.g., neural expression markers (e.g., NeuN), vector labels (e.g., GFP), and the like.

Data analysis including statistical testing, such as but not limited to mean±SEM of mechanical allodynia data. Treatment groups were compared to negative control groups using appropriate statistical tests.

Behavior data for cytomegalovirus (CMV)- and human synapsin 1 (hSyn1)-driven full-length human KCC2 in the SNL rat model is provided in FIG. 5. 20 μl each of CMV-driven full-length human KCC2 viral vector (CMV-FL hKCC2) with a titer of 1.79×10⁹ infectious particles/ml, hSyn1-driven full-length human KCC2 viral vector (hSyn1-FL hKCC2) with a titer of 1.26×10⁹ infectious particles/ml, and control vector with a titer of 1.86×10⁹ infectious particles/ml were infused into SNL rats at L5/L6 on day 15 or day 37 according to the experimental timelines shown in FIG. 4.

These data demonstrate that CMV-driven FL hKCC2 infused at day 15 significantly suppressed pain in the rat SNL model. In addition, hSyn1-driven FL hKCC2 infused at day 37 significantly suppressed pain in the rat SNL model. Moreover, hSyn1-driven FL hKCC2 infused at day 37 demonstrated more rapid pain suppression when compared to CMV-driven FL hKCC2 infused at day 15. Collectively, these data demonstrate effective pain suppression in an in vivo behavioral model of neuropathy following direct infusion of viral vectors of the present disclosure as compared to infusion with negative control.

Full-length, truncated and mutant forms of human KCC2 were compared as assessed in the behavioral setting of the rat SNL model. Rats were infused intrathecally at L5/L6 with 20 μl of control viral vector (1.86×10⁹ infectious particles/ml), CMV-FL hKCC2 (day 15) viral vector (1.79×10⁹ infectious particles/mi), CMV-driven truncated human KCC2 (CMV-trunc hKCC2; day 15) viral vector (1.66×10⁹ infectious particles/ml), and CMV-driven mutant human KCC2 (CMV-mut hKCC2; day 15) viral vector (2.30×10⁸ infectious particles/mi). Note that the amount of CMV-mut hKCC2 viral vector delivered represented 12.8% of the amount of CMV-FL hKCC2 viral vector delivered. Put another way, the amount of CMV-FL hKCC2 delivered was 7.78 times the amount of CMV-mut hKCC2 delivered.

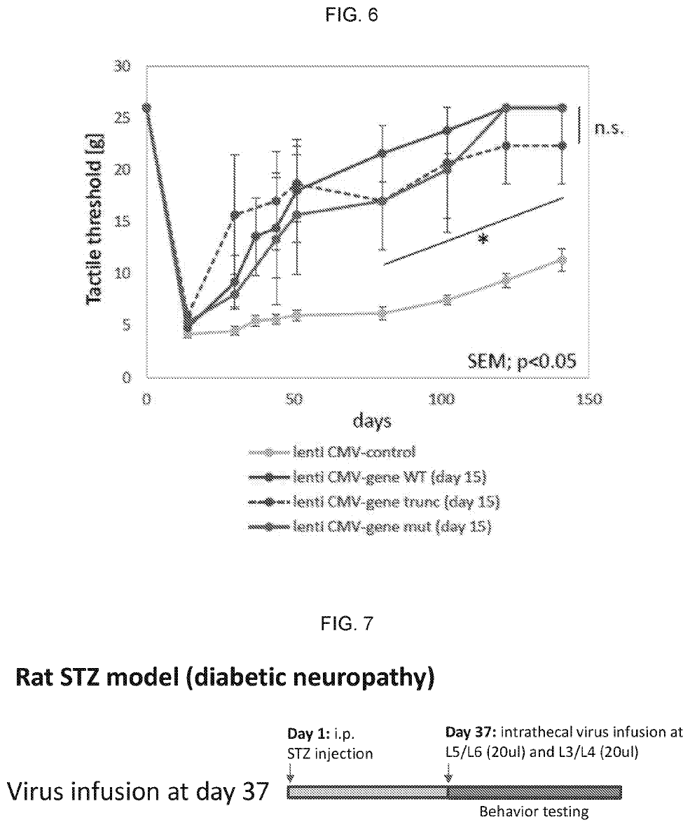

As shown in FIG. 6, significant differences in pain suppression were observed for each of the full-length, truncated and mutant KCC2 viral vector forms as compared to control. In addition, considering the reduced amount, at 12.8% of the amount of CMV-FL hKCC2 delivered, mut hKCC2 lentiviral particles demonstrated a trend of stronger pain suppression as compared to the full-length KCC2 construct. Moreover, the data obtained for truncated hKCC2, also showed a trend of stronger pain suppression as compared to full-length hKCC2. Collectively, these results demonstrate that lentiviral vectors encoding full-length, truncated and mutant forms of KCC2 all significantly suppressed pain in the SNL rat model as compared to negative control; with the truncated and mutant forms showing stronger pain suppression as compared to full-length hKCC2.

### Example 2: Evaluation of Therapeutic Activity in a Streptozotocin-Induced Diabetic Neuropathy Rat Model

This study demonstrates the effectiveness of agents of the present disclosure in reversing diabetic neuropathy, in the Streptozotocin (STZ)-induced neuropathic pain model. In the STZ model diabetes is induced by an injection of Streptozotocin (60 mg/kg) dissolved in citrate buffer (pH=6) into the tail vein of each rat. Animals are kept under a warming red lamp prior to injection and are slightly anaesthetized using ketamine/xylazine during the injection. The development of diabetes is confirmed by measuring the blood glucose levels (BGL) of animals included in the study.

The agents tested in this example included: lentivirus vector containing cytomegalovirus (CMV) promoter-driven full-length human KCC2 (CMV-FL hKCC2) and appropriate controls.

Agents were stored ready-to-use at −80° C., gently thawed, aliquoted and kept on ice prior to administration. Agents were delivered via intrathecal catheter at 40 μl per rat (20 μl at L5/L6 and 20 μl at L3/L4). After a small incision at back of neck and small opening in the atlanto-occipital membrane of the cisterna magna and dura, PE-10 catheter (9 cm) was inserted until L5+L6 spinal nerves and 20 μl of the test item was injected. Then, the catheter was slowly withdrawn 1 cm up to reach the L3+L4 spinal nerves area and 20 μl of the test item was injected. Three (3) minutes later, the catheter was slowly withdrawn. FIG. 7 provides an exemplary experimental timeline employed in the rat STZ model study.

STZ doses of 60 mg/kg, dosing volume 2.5 ml/kg, concentration of 24 mg/ml were prepared by adding 45 ml of deionized water to 5 ml of 10× citrate buffer, then adding 4.17 ml of prepared citrate buffer to a bottle containing 100 mg of STZ and vortexing. As an example, a rat weighing 200 g is injected IV with 0.5 ml of dissolved solution.

Animals were evaluated using various tests performed before and/or after agent or control administration, such tests included e.g., Von Frey tests, and BGL testing. Additional measurements were assessed during the study, including e.g., body weight measurement. Exemplary body weight measurements included measurement before SNL surgery for baseline values and once a week thereafter. Throughout the study, general clinical signs and observation were performed and recorded. Observations included changes in skin, fur, eyes, mucous membranes, occurrence of secretions and excretions (e.g. diarrhea) and autonomic activity (e.g., lacrimation, salivation, piloerection, pupil size, unusual respiratory pattern). Changes in gait, posture and response to handling, as well as the presence of abnormal behavior, tremors, convulsions, sleep and coma.

Animals having a BGL on study day 4 of greater than or equal to 300 mg/dl were considered diabetic and included in the study. Diabetic animals, with an average pain threshold of less than or equal to 8 g for both hind paws following Von Frey testing, were manually randomized to experimental groups based on similar mean pain thresholds. Von Frey testing was performed essentially as described above.

Data analysis including appropriate statistical testing, such as but not limited to mean±SEM of mechanical allodynia data. Treatment groups were compared to negative control groups using appropriate tests, including e.g., Students' T-test.

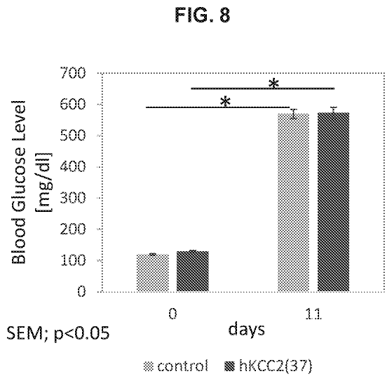

To assess the relevancy of the STZ model, control and hKCC2 treated rats were injected (i.p.) with STZ (day 1) and BGL was a measured at baseline (day 0) and post-PTZ treatment (day 11) timepoints. As shown in FIG. 8, streptozotocin (STZ)-mediated toxicity of insulin producing beta cells was confirmed by significantly increased blood glucose levels in the observed rats.

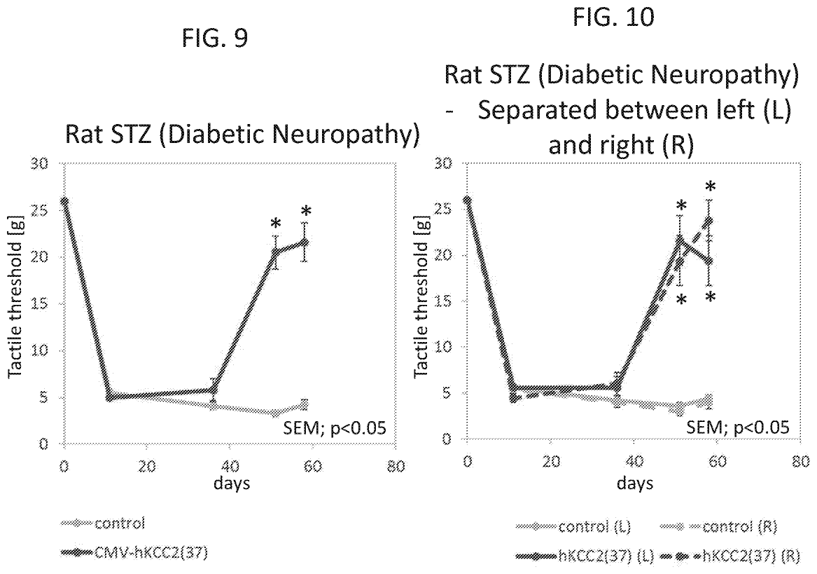

Behavior data demonstrated the effectiveness of CMV-driven full-length hKCC2 viral vector in suppressing diabetic neuropathy in the rat STZ model. For example, as compared to control viral vector (40 μl, infused at 1.86×10⁹ infectious particles/ml), CMV-FL hKCC2 viral vector (40 μl, infused at 1.79×10⁹ infectious particles/ml) administered intrathecally at L3/L4/L5/L6 significantly suppressed pain in the model (FIG. 9). Furthermore, separating assessments between left (L) and right (R) limbs of control treated and full-length hKCC2 treated rats further demonstrated significant suppression of pain in the corresponding limbs of the STZ diabetic rats (FIG. 10).

### Example 3: Method for Patient Stratification Employing Detection of Human KCC2 as Biomarker in Lumbar CSF

Detection of human KCC2 in CSF was investigated as a method for patient identification and/or stratification, e.g., prior to treatment according to the methods as described herein. Lumbar CSF samples were obtained from healthy subjects and pain subjects. The pain subject samples were sub-grouped based according to severity, including one group with a pain score of 7 out of 12 and another group with a pain score of 5 out of 12. Samples were blinded and analyzed by ProteinSimple microcapillary in duplicate (5 μg concentrated sample) and quantification was performed using a Protein Quant kit.

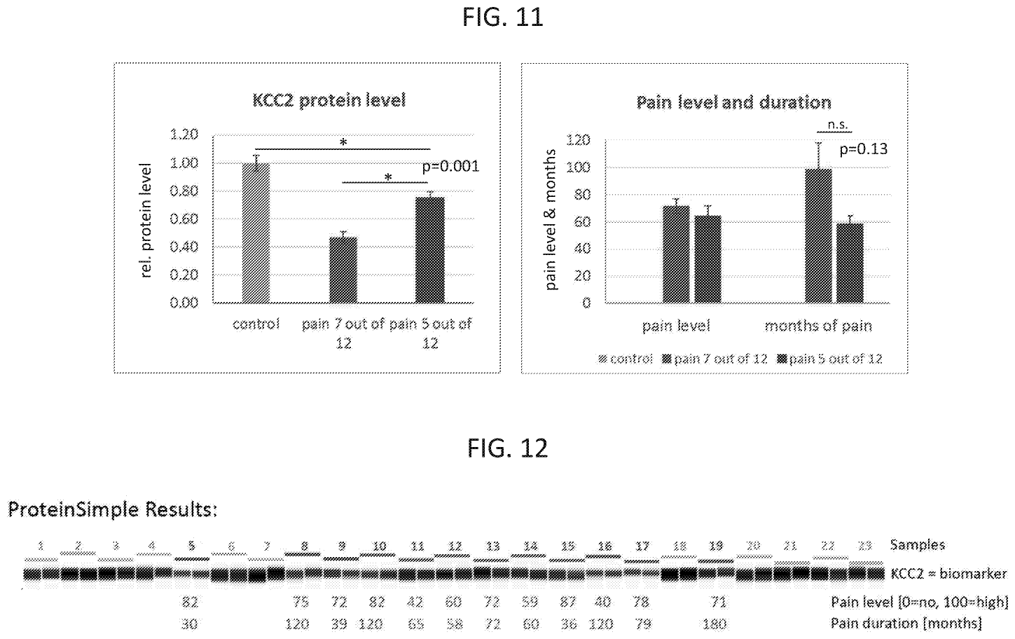

This example demonstrates that hKCC2 protein can be detected in lumbar CSF samples taken from healthy and pain patients using a ProteinSimple-based microcapillary detection method. In addition, as shown in FIG. 11, hKCC2 protein level is significantly suppressed in human lumbar CSF samples from pain patients when compared to healthy control CSF samples, regardless of pain level sub-stratification. The individual ProteinSimple results are provided in FIG. 12, with the pain level (on a scale of 0 to 100, where zero=no pain) and pain duration (in months) of each subject from which the samples were derived indicated. Collectively, these data demonstrate that hKCC2 protein is an effective CSF biomarker to stratify patients for treatment of KCC2-mediated diseases, including pain and others, including where such treatments may include those described herein.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A composition comprising:
a viral vector comprising a viral backbone nucleic acid comprising a sequence encoding a full-length human K-Cl cotransporter 2 (KCC2) polypeptide; and
a pharmaceutically acceptable diluent.

2. The composition according to claim 1, wherein the viral vector is a lentiviral vector.

3. The composition according to claim 1, wherein the viral vector is an adeno-associated virus (AAV) vector.

4. The composition according to claim 1, wherein the viral backbone nucleic acid is a human immunodeficiency (HIV) backbone, an equine infectious anemia virus (EIAV) backbone, or an AAV backbone.

5. The composition according to claim 1, wherein the viral vector is present in the diluent at a concentration of 1×10⁷ to 1×10¹⁵ infectious particles per milliliter.

6. The composition according to claim 1, wherein the composition is formulated in unit dosage form in a delivery device.

7. The composition according to claim 6, wherein the delivery device comprises 10 microliters to 1 milliliter of the composition.

8. The composition according to claim 6, wherein the delivery device is an injection device configured for intrathecal or intraspinal administration.

9. The composition according to claim 1, wherein the composition is formulated for in vivo delivery for the treatment of pain.

10. A composition comprising a gene therapy vector comprising a nucleic acid sequence encoding a modified K-Cl cotransporter 2 (KCC2) polypeptide.

11.-31. (canceled)

32. A method of treating a mammalian subject for a pain condition, the method comprising administering to the subject a gene therapy agent effective to cause the subject to express an effective amount of a modified K-Cl cotransporter 2 (KCC2) polypeptide having enhanced activity relative to wild-type KCC2.

33. The method according to claim 32, wherein expressing the modified KCC2 polypeptide comprises expressing a heterologous KCC2 polypeptide encoded by the gene therapy agent.

34. The method according to claim 32, wherein the gene therapy agent comprises a viral vector according to any of claims 1 to 9 or a gene therapy vector according to any of claims 10 to 23.

35. The method according to claim 32, wherein expressing the modified KCC2 polypeptide comprises editing an endogenous KCC2 locus of the subject to encode the modified KCC2 polypeptide.

36. The method according to claim 35, wherein the endogenous KCC2 locus is edited to encode a modified KCC2 polypeptide comprising an N-terminal truncation relative to a wild-type KCC2 polypeptide, one or more substitution mutations relative to a wild-type KCC2 polypeptide, or both.

37. The method according to claim 32, wherein the administering is effective to cause the subject to express an effective amount of the modified KCC2 in dorsal horn spinal cord neurons of the subject.

38. The method according to claim 32, wherein the pain condition comprises peripheral nerve damage.

39. The method according to claim 38, wherein the pain condition is osteoarthritis.

40. The method according to claim 38, wherein the pain condition is a peripheral neuropathy.

41. The method according to claim 40, wherein the peripheral neuropathy comprises diabetic neuropathy.

42. The method according to claim 32, wherein the pain condition does not comprise spinal cord injury.

43. The method according to claim 42, wherein the pain condition does not comprise central nervous system injury.

44.-70. (canceled)


SEQUENCE LISTING (5 sequences)

SEQ ID NO: 1 - protein, 1139 aa, Homo sapiens
MSRRFTVTSLPPAGPARSPDPESRRHSVADPRHLPGEDVKGDGNPKESSPFINSTDTEKGKEYDGKNMALFEEEMDTSPMVSSLLSGLANYTNLPQGSREHEEAENNEGGKKKPVQAPRMGTFMGVYLPCLQNIFGVILFLRLTWVVGIAGIMESFCMVFICCSCTMLTAISMSAIATNGVVPAGGSYYMISRSLGPEFGGAVGLCFYLGTTFAGAMYILGTIEILLAYLFPAMAIFKAEDASGEAAAMLNNMRVYGTCVLTCMATVVFVGVKYVNKFALVFLGCVILSILAIYAGVIKSAFDPPNFPICLLGNRTLSRHGFDVCAKLAWEGNETVTTRLWGLFCSSRFLNATCDEYFTRNNVTEIQGIPGAASGLIKENLWSSYLTKGVIVERSGMTSVGLADGTPIDMDHPYVFSDMTSYFTLLVGIYFPSVTGIMAGSNRSGDLRDAQKSIPTGTILAIATTSAVYISSVVLFGACIEGVVLRDKFGEAVNGNLVVGTLAWPSPWVIVIGSFFSTCGAGLQSLTGAPRLLQAISRDGIVPFLQVFGHGKANGEPTWALLLTACICEIGILIASLDEVAPILSMFFLMCYMFVNLACAVQTLLRTPNWRPRFRYYHWTLSFLGMSLCLALMFICSWYYALVAMLIAGLIYKYIEYRGAEKEWGDGIRGLSLSAARYALLRLEEGPPHTKNWRPQLLVLVRVDQDQNVVHPQLLSLTSQLKAGKGLTIVGSVLEGTFLENHPQAQRAEESIRRLMEAEKVKGFCQVVISSNLRDGVSHLIQSGGLGGLQHNTVLVGWPRNWRQKEDHQTWRNFIELVRETTAGHLALLVTKNVSMFPGNPERFSEGSIDVWWIVHDGGMLMLLPFLLRHHKVWRKCKMRIFTVAQMDDNSIQMKKDLTTFLYHLRITAEVEVVEMHESDISAYTYEKTLVMEQRSQILKQMHLTKNEREREIQSITDESRGSIRRKNPANTRLRLNVPEETAGDSEEKPEEEVQLIHDQSAPSCPSSSPSPGEEPEGEGETDPEKVHLTWTKDKSVAEKNKGPSPVSSEGIKDFFSMKPEWENLNQSNVRRMHTAVRLNEVIVKKSRDAKLVLLNMPGPPRNRNGDENYMEFLEVLTEHLDRVMLVRGGGREVITIYS

SEQ ID NO: 2 - protein, 1116 aa, Homo sapiens
MLNNLTDCEDGDGGANPGDGNPKESSPFINSTDTEKGKEYDGKNMALFEEEMDTSPMVSSLLSGLANYTNLPQGSREHEEAENNEGGKKKPVQAPRMGTFMGVYLPCLQNIFGVILFLRLTWVVGIAGIMESFCMVFICCSCTMLTAISMSAIATNGVVPAGGSYYMISRSLGPEFGGAVGLCFYLGTTFAGAMYILGTIEILLAYLFPAMAIFKAEDASGEAAAMLNNMRVYGTCVLTCMATVVFVGVKYVNKFALVFLGCVILSILAIYAGVIKSAFDPPNFPICLLGNRTLSRHGFDVCAKLAWEGNETVTTRLWGLFCSSRFLNATCDEYFTRNNVTEIQGIPGAASGLIKENLWSSYLTKGVIVERSGMTSVGLADGTPIDMDHPYVFSDMTSYFTLLVGIYFPSVTGIMAGSNRSGDLRDAQKSIPTGTILAIATTSAVYISSVVLFGACIEGVVLRDKFGEAVNGNLVVGTLAWPSPWVIVIGSFFSTCGAGLQSLTGAPRLLQAISRDGIVPFLQVFGHGKANGEPTWALLLTACICEIGILIASLDEVAPILSMFFLMCYMFVNLACAVQTLLRTPNWRPRFRYYHWTLSFLGMSLCLALMFICSWYYALVAMLIAGLIYKYIEYRGAEKEWGDGIRGLSLSAARYALLRLEEGPPHTKNWRPQLLVLVRVDQDQNVVHPQLLSLTSQLKAGKGLTIVGSVLEGTFLENHPQAQRAEESIRRLMEAEKVKGFCQVVISSNLRDGVSHLIQSGGLGGLQHNTVLVGWPRNWRQKEDHQTWRNFIELVRETTAGHLALLVTKNVSMFPGNPERFSEGSIDVWWIVHDGGMLMLLPFLLRHHKVWRKCKMRIFTVAQMDDNSIQMKKDLTTFLYHLRITAEVEVVEMHESDISAYTYEKTLVMEQRSQILKQMHLTKNEREREIQSITDESRGSIRRKNPANTRLRLNVPEETAGDSEEKPEEEVQLIHDQSAPSCPSSSPSPGEEPEGEGETDPEKVHLTWTKDKSVAEKNKGPSPVSSEGIKDFFSMKPEWENLNQSNVRRMHTAVRLNEVIVKKSRDAKLVLLNMPGPPRNRNGDENYMEFLEVLTEHLDRVMLVRGGGREVITIYS

SEQ ID NO: 3 - protein, 1100 aa, Homo sapiens
MGDGNPKESSPFINSTDTEKGKEYDGKNMALFEEEMDTSPMVSSLLSGLANYTNLPQGSREHEEAENNEGGKKKPVQAPRMGTFMGVYLPCLQNIFGVILFLRLTWVVGIAGIMESFCMVFICCSCTMLTAISMSAIATNGVVPAGGSYYMISRSLGPEFGGAVGLCFYLGTTFAGAMYILGTIEILLAYLFPAMAIFKAEDASGEAAAMLNNMRVYGTCVLTCMATVVFVGVKYVNKFALVFLGCVILSILAIYAGVIKSAFDPPNFPICLLGNRTLSRHGFDVCAKLAWEGNETVTTRLWGLFCSSRFLNATCDEYFTRNNVTEIQGIPGAASGLIKENLWSSYLTKGVIVERSGMTSVGLADGTPIDMDHPYVFSDMTSYFTLLVGIYFPSVTGIMAGSNRSGDLRDAQKSIPTGTILAIATTSAVYISSVVLFGACIEGVVLRDKFGEAVNGNLVVGTLAWPSPWVIVIGSFFSTCGAGLQSLTGAPRLLQAISRDGIVPFLQVFGHGKANGEPTWALLLTACICEIGILIASLDEVAPILSMFFLMCYMFVNLACAVQTLLRTPNWRPRFRYYHWTLSFLGMSLCLALMFICSWYYALVAMLIAGLIYKYIEYRGAEKEWGDGIRGLSLSAARYALLRLEEGPPHTKNWRPQLLVLVRVDQDQNVVHPQLLSLTSQLKAGKGLTIVGSVLEGTFLENHPQAQRAEESIRRLMEAEKVKGFCQVVISSNLRDGVSHLIQSGGLGGLQHNTVLVGWPRNWRQKEDHQTWRNFIELVRETTAGHLALLVTKNVSMFPGNPERFSEGSIDVWWIVHDGGMLMLLPFLLRHHKVWRKCKMRIFTVAQMDDNSIQMKKDLTTFLYHLRITAEVEVVEMHESDISAYTYEKTLVMEQRSQILKQMHLTKNEREREIQSITDESRGSIRRKNPANTRLRLNVPEETAGDSEEKPEEEVQLIHDQSAPSCPSSSPSPGEEPEGEGETDPEKVHLTWTKDKSVAEKNKGPSPVSSEGIKDFFSMKPEWENLNQSNVRRMHTAVRLNEVIVKKSRDAKLVLLNMPGPPRNRNGDENYMEFLEVLTEHLDRVMLVRGGGREVITIYS

SEQ ID NO: 4 - protein, 1100 aa, Artificial Sequence
  synthetic sequence
MGDGNPKESSPFINSTDTEKGKEYDGKNMALFEEEMDTSPMVSSLLSGLANYTNLPQGSREHEEAENNEGGKKKPVQAPRMGTFMGVYLPCLQNIFGVILFLRLTWVVGIAGIMESFCMVFICCSCTMLTAISMSAIATNGVVPAGGSYYMISRSLGPEFGGAVGLCFYLGTTFAGAMYILGTIEILLAYLFPAMAIFKAEDASGEAAAMLNNMRVYGTCVLTCMATVVFVGVKYVNKFALVFLGCVILSILAIYAGVIKSAFDPPNFPICLLGNRTLSRHGFDVCAKLAWEGNETVTTRLWGLFCSSRFLNATCDEYFTRNNVTEIQGIPGAASGLIKENLWSSYLTKGVIVERSGMTSVGLADGTPIDMDHPYVFSDMTSYFTLLVGIYFPSVTGIMAGSNRSGDLRDAQKSIPTGTILAIATTSAVYISSVVLFGACIEGVVLRDKFGEAVNGNLVVGTLAWPSPWVIVIGSFFSTCGAGLQSLTGAPRLLQAISRDGIVPFLQVFGHGKANGEPTWALLLTACICEIGILIASLDEVAPILSMFFLMCYMFVNLACAVQTLLRTPNWRPRFRYYHWTLSFLGMSLCLALMFICSWYYALVAMLIAGLIYKYIEYRGAEKEWGDGIRGLSLSAARYALLRLEEGPPHTKNWRPQLLVLVRVDQDQNVVHPQLLSLTSQLKAGKGLTIVGSVLEGTFLENHPQAQRAEESIRRLMEAEKVKGFCQVVISSNLRDGVSHLIQSGGLGGLQHNTVLVGWPRNWRQKEDHQTWRNFIELVRETTAGHLALLVTKNVSMFPGNPERFSEGSIDVWWIVHDGGMLMLLPFLLRHHKVWRKCKMRIFTVAQMDDNSIQMKKDLTTFLYHLRITAEVEVVEMHESDISAYTYEKALVMEQRSQILKQMHLTKNEREREIQSIDDEDRGSIRRKNPANTRLRLNVPEETAGDSEEKPEEEVQLIHDQSAPSCPSSSPSPGEEPEGEGETDPEKVHLAWTKDKSVAEKNKGPSPVSSEGIKDFFSMKPEWENLNQSNVRRMHTAVRLNEVIVKKSRDAKLVLLNMPGPPRNRNGDENYMEFLEVLTEHLDRVMLVRGGGREVITIYS

SEQ ID NO: 5 - protein, 1116 aa, Artificial Sequence
  synthetic sequence
MLNNLTDCEDGDGGANPGDGNPKESSPFINSTDTEKGKEYDGKNMALFEEEMDTSPMVSSLLSGLANYTNLPQGSREHEEAENNEGGKKKPVQAPRMGTFMGVYLPCLQNIFGVILFLRLTWVVGIAGIMESFCMVFICCSCTMLTAISMSAIATNGVVPAGGSYYMISRSLGPEFGGAVGLCFYLGTTFAGAMYILGTIEILLAYLFPAMAIFKAEDASGEAAAMLNNMRVYGTCVLTCMATVVFVGVKYVNKFALVFLGCVILSILAIYAGVIKSAFDPPNFPICLLGNRTLSRHGFDVCAKLAWEGNETVTTRLWGLFCSSRFLNATCDEYFTRNNVTEIQGIPGAASGLIKENLWSSYLTKGVIVERSGMTSVGLADGTPIDMDHPYVFSDMTSYFTLLVGIYFPSVTGIMAGSNRSGDLRDAQKSIPTGTILAIATTSAVYISSVVLFGACIEGVVLRDKFGEAVNGNLVVGTLAWPSPWVIVIGSFFSTCGAGLQSLTGAPRLLQAISRDGIVPFLQVFGHGKANGEPTWALLLTACICEIGILIASLDEVAPILSMFFLMCYMFVNLACAVQTLLRTPNWRPRFRYYHWTLSFLGMSLCLALMFICSWYYALVAMLIAGLIYKYIEYRGAEKEWGDGIRGLSLSAARYALLRLEEGPPHTKNWRPQLLVLVRVDQDQNVVHPQLLSLTSQLKAGKGLTIVGSVLEGTFLENHPQAQRAEESIRRLMEAEKVKGFCQVVISSNLRDGVSHLIQSGGLGGLQHNTVLVGWPRNWRQKEDHQTWRNFIELVRETTAGHLALLVTKNVSMFPGNPERFSEGSIDVWWIVHDGGMLMLLPFLLRHHKVWRKCKMRIFTVAQMDDNSIQMKKDLTTFLYHLRITAEVEVVEMHESDISAYTYEKALVMEQRSQILKQMHLTKNEREREIQSIDDEDRGSIRRKNPANTRLRLNVPEETAGDSEEKPEEEVQLIHDQSAPSCPSSSPSPGEEPEGEGETDPEKVHLAWTKDKSVAEKNKGPSPVSSEGIKDFFSMKPEWENLNQSNVRRMHTAVRLNEVIVKKSRDAKLVLLNMPGPPRNRNGDENYMEFLEVLTEHLDRVMLVRGGGREVITIYS